(19) United States
(12) Patent Application Publication
(10) Pub. No.: US 2019/0391167 A1
(43) Pub. Date: Dec. 26, 2019

(54) METHOD FOR PREDICTING THE DEVELOPMENT OF TYPE 2 DIABETES

(71) Applicants: The Governing Council of the University of Toronto, Toronto (CA); Kaiser Permanente Northern California, Oakland, CA (US)

(72) Inventors: Michael WHEELER, Toronto (CA); Amina ALLALOU, Montreal (CA); Ying LIU, Thornhill (CA); Kacey PRENTICE, Allston, MA (US); Feihan DAI, North York (CA); Ming ZHANG, Toronto (CA); Erica GUNDERSON, Oakland, CA (US); Amarnadh NALLA, Copenhagen (DK)

(21) Appl. No.: 16/302,287

(22) PCT Filed: May 16, 2017
(86) PCT No.: PCT/CA2017/050581

§ 371 Date: Nov. 16, 2018

Related U.S. Application Data
(60) Provisional application No. 62/337,046, filed on May 16, 2016.

(51) Int. Cl. G01N 33/92; G16B 5/20; G16B 50/00; G01N 33/68

(52) U.S. Cl. CPC G01N 33/92; G16B 5/20; G16B 50/00; G01N 33/6806; G01N 2800/52; G01N 2560/00; G01N 2405/08; G01N 2800/50; G01N 2800/042

(57) ABSTRACT

A method of predicting progression of gestational diabetes (GDM) to Type 2 diabetes (T2D) in a subject is provided. The method comprises: analyzing a biological sample of a subject to determine levels of a plurality of metabolites in the sample, wherein the plurality of metabolites comprises one or more of PCaeC40:5 and SM(OH)C14:1 and at least two metabolites set forth in Table 3, 4 and/or 6; and comparing the determined levels of the plurality of metabolites in the sample to a corresponding plurality of reference levels in order to predict progression of GDM to T2D in the subject.

### CROSS REFERENCE TO PRIOR APPLICATIONS

This application claims priority under the Paris Convention to U.S. Provisional Patent Application 62/337,046, filed May 16, 2016, which is incorporated herein by reference as if set forth in its entirety.

### FIELD OF THE DESCRIPTION

The present description relates generally to the detection of biomarkers and the prediction of type 2 diabetes (T2D). More specifically, the present description relates to methods and kits for predicting T2D in patients having gestational diabetes (GDM) and methods of treating same.

### BACKGROUND OF THE DESCRIPTION

Gestational diabetes mellitus (GDM) occurs in 3-14% of pregnancies and 20-50% of women with GDM develop type 2 diabetes (T2D) within 5 years of the index pregnancy (1; 2). The American Diabetes Association (ADA) thus recommends T2D screening at 6-12 weeks postpartum and every 1 to 3 years thereafter via testing fasting plasma glucose (FPG), 2-hr 75 g oral glucose tolerance test (OGTT), or hemoglobin A1c for women in this high risk population (3). However, screening of women post-GDM pregnancy is sub-optimal, with low compliance rates of 16-19% (4; 5), although integrated health care systems report 60% screening (2). Reasons for those low rates include logistical difficulties of administering an oral glucose tolerance test (OGTT), fear of receiving a diagnosis of diabetes (6) and failure to attend the post-partum follow-up exam (7). Further, many women with a previous GDM pregnancy hold a faulty low risk perception of T2D incidence (8; 9). Several risk scores have been developed for T2D (10; 11), none of them consider a history of GDM diagnosis. Prediction of T2D in women with a previous GDM pregnancy is important for individual risk stratification and/or early prevention following delivery.

### SUMMARY OF THE DESCRIPTION

In an aspect, a method of predicting progression of gestational diabetes (GDM) to Type 2 diabetes (T2D) in a subject is provided. The method comprises: analyzing a biological sample of a post-partum subject to determine levels of a plurality of metabolites in the sample, wherein the plurality of metabolites comprises one or more of PCaeC40:5 and SM(OH)C14:1 and at least two metabolites set forth in Table 3, 4 and/or 6; and comparing the determined levels of the plurality of metabolites in the sample to a corresponding plurality of reference levels in order to predict progression of GDM to T2D in the subject.

In an embodiment, the plurality of metabolites comprises at least one amino selected from: 2-Aminoadipic acid, Gly, Arg, Gln, His, Ile, Leu, Met, Orn, Phe, PAG, Pro, Ser, Thr, Trp, Tyr, Val, and xLeu.

In an embodiment, the amino acid is one or more branched chain amino acid selected from: 2-Aminoadipic acid, Gly, Ile, Leu, Thr, Trp, Tyr, Val, xLeu, preferably xLeu and Val.

In an embodiment, the plurality of metabolites comprises at least one sphingomyelin (SM) species selected from: SM(OH)C14:1, SM (OH) C16:1, SM (OH) C22:1, SM (OH) C22:2, SM (OH) C24:1, SM C16:0, SM C16:1, SM C18:0, SM C18:1, SM C20:2, SM C24:0, SM C24:1, preferably SM(OH)C14:1.

In an embodiment, the plurality of metabolites comprises at least one lipid/fatty acid selected from: Myristic acid (C14:0), Palmitic acid (C16:0), Hexadecenoic acid (C16:1 n-7), Palmitoleic acid (C16:1 n-9), Stearic acid (C18:0), Oleic Acid & Vaccenic Acid (C18:1 n-9, n-7), Linoleic acid (C18:2), Alpha-linolenic acid (C18:3), Eicosenoic acid (C20:1), Arachidonic acid (C20:4), Eicosapentaenoic acid (C20:5), Docosapentaenoic acid (C22:5), Docosahexaenoic acid (C22:6), preferably, Palmitoleic acid (C16:1 n9).

In an embodiment, the plurality of metabolites comprises at least one ketone, preferably beta-hydroxybutyrate.

In an embodiment, the plurality of metabolites comprises one or more of PCaeC40:5 and SM(OH)C14:1, and two or more of 2-aminoadipic acid, Ile, Leu, Thr, Trp, Tyr, Val, xLeu, Hexose, AC3, Gly, SM (OH) C16:1, SM (OH) C22:2, SM C18:0, SM C18:1, SM C20:2, SM C24:1, PC ae C42:5, PC ae C44:5, AC10, and palmitoleic acid (C16:1 n9).

In an embodiment, the plurality of reference levels are indicative of levels of the plurality of metabolites in subjects whose GDM did not progress to T2D.

In an embodiment, a determined increase of one or more of 2-aminoadipic acid, Ile, Leu, Thr, Trp, Tyr, Val, xLeu, Hexose and AC3 relative to the respective plurality of reference levels is indicative of progression of GDM to T2D.

In an embodiment, a determined decrease of one or more of Gly, SM(OH)C14:1, SM (OH) C16:1, SM (OH) C22:2, SM C18:0, SM C18:1, SM C20:2, SM C24:1, PC ae C40:5, PC ae C42:5, PC ae C44:5, AC10 and palmitoleic acid (C16:1 n9) relative to the respective plurality of reference levels is indicative of progression of GDM to T2D.

In an embodiment, the plurality of metabolites comprises PC ae C40:5, SM (OH) C14:1, hexoses, Val, Leu, and Ile.

In an embodiment, the progression of GDM to T2D is within 0-5 years of delivery, preferably within 0-2 years of delivery.

In an embodiment, the biological sample comprises a plasma sample, preferably a fasting plasma sample.

In an embodiment, the plasma sample is obtained from the subject at 6-9 weeks post-partum.

In an embodiment, the determining is by one or more of LC-MS/MS, GC-MS, ELISA while Fasting (FPG) and antibody detection.

In an embodiment, the method further comprises: treating the subject based on a result of the comparison, wherein the treatment comprises one or more of: diet regimen, exercise regimen, blood sugar monitoring, insulin therapy, and medication.

In an embodiment, the determination of the levels of the plurality of metabolites in the sample comprises detecting a derivative of one or more of the plurality of metabolites.

In an aspect, a computer-implemented method of predicting progression of gestational diabetes (GDM) to Type 2 diabetes (T2D) in a subject is provided. The method comprises: measuring in a biological sample from a post-partum subject an incident type 2 diabetes (T2D) biomarker panel, wherein the incident T2D biomarker panel comprises one or more of PCaeC40:5 and SM(OH)C14:1 and at least two biomarkers set forth in Table 3, 4 and/or 6; applying the measured incident T2D biomarker panel from the subject against a database of measured T2D biomarker panels from control subjects, wherein the database is stored on a computer system; and determining that the subject has an increased risk of progression to T2D by measuring difference in the incident T2D biomarker panel relative to measured incident T2D biomarker panels from control subjects.

In an aspect, a non-transitory computer readable storage medium with an executable program stored thereon is provided. The program comprises instructions for evaluating a subject's risk for progressing from GDM to T2D, and wherein the program instructs a microprocessor to perform one or more of the steps of any one of the methods provided herein.

In an aspect, a computer system is provided. The system comprises: a database including records comprising reference metabolite profiles associated with clinical outcomes, each reference profile comprising the levels of a set of metabolites listed in Table 3, 4, and/or 6; a user interface capable of receiving and/or inputting a selection of metabolite levels of a set of metabolites, the set of metabolites listed in Table 3, 4, and/or 6 for use in comparing to the metabolite reference profiles in the database; and an output that displays a prediction of clinical prognosis according to the levels of the set of metabolites.

In an embodiment, the computer system is for performing one or more of the methods provided herein.

### BRIEF DESCRIPTION OF THE DRAWINGS

The features of the invention will become more apparent in the following detailed description in which reference is made to the appended drawings wherein:

FIGS. 1a and 1b depict study design and metabolic assay work flow.

FIG. 1a depicts study design of the SWIFT prospective cohort, a total of 1035 women diagnosed with GDM were enrolled at 6-9 weeks post-partum (baseline) and screened via 2-hr 75 g OGTTs.

FIG. 1b depicts work flow of metabolomics assay, in which a total of 182 metabolites were assayed in plasma from V1 (baseline) using LC-MS/MS, GC-MS and ELISA.

FIGS. 2a-b depict a decision tree and ROC for an embodiment of the prediction of Incident T2D.

FIG. 2a depicts a decision tree by J48 based on the combined AUC and F-score of all algorithms; the grey boxes indicate the metabolite chosen for the node and the clear numbered boxes indicate the concentration threshold in μM for PC ae C40:5, BCAA and SM (OH) C14:1 and mM for hexoses.

FIG. 2b depicts an ROC of the J48 algorithm on the training and testing set, performing with discriminative power 0.830 (p<0.000001) and 0.769 (p<0.0001), respectively, which is greater than FPG alone 0.724 (p<0.0001) and 0.706 (p<0.01), as well as 2hPG alone 0.726 (p<0.000001) and 0.661 (p<0.05), respectively (data presented in AUC).

FIGS. 3a-b depicts Venn diagrams and contingency tables comparison of model predictions of future diabetes.

FIG. 3a depicts Venn diagrams of correct and incorrect predictions of the testing data set for all patients, only incident T2D and only non-T2D (Non) patients are shown; correct prediction numbers are underlined (green), incorrect predictions are not underlined (red).

FIG. 3b depicts contingency tables of the three different models against the testing data set; columns are known group labels and rows are predicted group labels; the metabolite model (left) shows the higher precision (double underline) and specificity (single underline) compared to the glucose model; the combined model (right) has overall poorer sensitivity (no underline) and specificity compared to both the metabolite and glucose models alone.

### DETAILED DESCRIPTION OF THE NON-LIMITING EXEMPLARY EMBODIMENTS

Herein the invention will be described in conjunction with certain representative embodiments. However, it will be understood that the invention is not limited to those embodiments. One skilled in the art will recognize that various methods and materials similar or equivalent to those described herein may be used in the practice of the present disclosure.

Unless defined otherwise, all technical and scientific terms used herein generally have the same meaning as commonly understood by one of ordinary skill in the art to which this invention belongs.

All publications, published patent documents, and patent applications cited herein are hereby incorporated by reference as if set forth in their entirety.

As used herein, “biological sample” and “sample” refer to any material, obtained from an individual, which may contain a plurality of metabolites as provided herein. This includes blood (including whole blood, plasma, and/or serum). This also includes experimentally separated fractions of all of the preceding. Any suitable methods for obtaining a biological sample can be employed and the sample may be processed in any suitable manner after being obtained from the individual.

As used herein, “marker” and “biomarker” are used to refer to a target molecule that indicates a normal or abnormal process in an individual. More specifically, a “marker” or “biomarker” is metabolic parameter associated with the a specific physiological state or process, whether normal or abnormal, such as a metabolite. Biomarkers are detectable and measurable by a variety of methods including laboratory assays, such as MS-based assays.

As used herein, “biomarker level”, “metabolite level”, and “level” refer to a measurement that is made using any analytical method for detecting the biomarker in a biological sample and that indicates the presence, absence, absolute amount or concentration, relative amount or concentration, a ratio of measured levels, of, for, or corresponding to the biomarker in the biological sample. The exact nature of the “level” depends on the specific design and components of the particular analytical method employed to detect the biomarker.

A “reference level” or “control level” of a biomarker refers to the level of the biomarker in the same sample type from an individual (or individuals) whose GDM did not progress to T2D. A “control level” of a biomarker need not be determined each time the present methods are carried out, and may be a previously determined level that is used as a reference or threshold to determine whether the level in a particular sample is higher or lower than a normal “control” level. In some embodiments, a control level in a method described herein is the level that has been observed in one or more subjects with non-progressive GDM. In some embodiments, a control level in a method described herein is the average or mean level, optionally plus or minus a statistical variation, that has been observed in a plurality of subjects with non-progressive GDM.

As used herein, “subject” and “individual” are used interchangeably to refer to a test subject or patient. The individual is a mammal that may be human or non-human. In various embodiments, the individual is a human. A healthy or normal individual is an individual in which GDM does not progress to T2D. As used herein, a “subject with gestational diabetes (GDM)” refers to a subject that was been diagnosed with GDM during pregnancy. GDM may have been diagnosed using a known method.

As used herein, “progressive GDM” refers to GDM that is progressing towards T2D.

TABLE 1
Abbreviations used herein:

| | |
|---|---|
| 2-AAA | 2-aminoadipic acid |
| 2hPG | 2 hour post-load plasma glucose after 75 gram OGTT |
| AC | Acylcarnitines |
| ADA | American Diabetes Association |
| Arg | Arginine |
| AUC | Area under the curve |
| BCAA | Branched chain amino acids |
| BMI | Body mass index |
| CV | Co-efficient variation |
| DT | J48 decision tree |
| FFA | Free fatty acids |
| FPG | Fasting plasma glucose |
| FPIC | Female plasma internal standard |
| GDM | Gestational diabetes mellitus |
| Gln | Glutamine |
| Gly | Glycine |
| HDL | High density lipoprotein |
| His | Histidine |
| Ile | Isoleucine |
| Leu | Leucine |
| LR | Logistic regression |
| LLOQ | Lower limit of quantification |
| LOD | Limit of detection |
| LPC | lysophosphatidylcholine |
| Met | Methionine |
| NB | Naïve Bayes |
| NGT | Normal glucose tolerant |
| non-T2D | Did not develop type 2 diabetes |
| OGTT | Oral glucose tolerance test |
| Orn | Ornithine |
| PAG | Phenyl acetyl glutamine |
| PC | Phosphatidylcholine |
| PG | Plasma glucose |
| Phe | Phenylalanine |
| Pro | Proline |
| QNT | Quantitative |
| ROC | Receiver operating curve |
| Se | Sensitivity |
| Ser | Serine |
| SM | Sphingolipids |
| Sp | Specificity |
| SQ | Semi-quantitative |
| SWIFT | Study of Women, Infant Feeding, and Type 2 |
| | diabetes mellitus after |
| GDM | Gestational Diabetes |
| T2D | Type 2 diabetes |
| Thr | Threonine |
| Try | Tryptophan |
| Try | Tyrosine |
| Val | Valine |
| Xleu | xleucine |

The present disclosure is directed to biomarkers, methods, systems, and media for predicting progression from GDM to T2D in a subject and treatment of those subjects predicted to progress from GDM to T2D.

As described herein, the inventors have determined an in vitro method for predicting progression from gestational diabetes (GDM) to Type 2 Diabetes (T2D) in a subject. The method involves measuring the levels of a plurality of metabolites in a biological sample from the subject. In some embodiments, measuring the levels of a plurality of metabolites in a biological sample allows identification of a metabolic signature for the biological sample, which may be predictive of progression from GDM to T2D.

The present inventors used a metabolomics approach that implements advanced machine learning methods as a tool to identify early diagnostic biomarkers that have predictive abilities for complex pathologies, such as diabetes, which is a heterogeneous disorder of glucose metabolism that can have diverse root cause across various racial and ethnic subgroups (12). As described in further detail in the Examples, the inventors measured numerous metabolites in stored frozen fasting plasma samples drawn at 6-9 weeks post-partum under standardized research protocols from women with recent GDM without diabetes via the 2-hr 75 g OGTT and in whom annual follow-up screening (2-hr 75 g OGTT) was conducted to assess new onset of T2D within two years.

Previous metabolomic investigations of T2D in the general population have revealed significant differences between diabetic patients and normal glucose tolerant (NGT) controls (13-22), although the majority of these were cross-sectional studies of T2D prevalence. One study involved lipodomic analysis and evaluation of risk of T2D among women of northern European ancestry with previous GDM (23). In this study, clinical variables, such as, for example, those set forth in Table 2, combined with lipid species predicted 21 cases of T2D during 8.5 years of follow-up with over 80% accuracy. However, this signature has not been independently validated, or tested among other ethnicities. The study provided herein represents the first metabolomics study of the transition from GDM to T2D and offers a quantitative measure of risk.

In an aspect, four or more biomarkers are provided herein for use in various combinations to predict progression from GDM to T2D in a subject. As described in detail below, exemplary embodiments include the biomarkers provided in Tables 4 and/or 6, one or more of which may be measured, for example, using a mass spectrometry (MS)-based assay. The biomarkers in Table 3 may also be useful for predicting progression from GDM to T2D in a subject.

In some embodiments, a method comprises detecting at least four biomarkers, at least five biomarkers, at least six biomarkers, at least seven biomarkers, at least eight biomarkers, at least nine biomarkers, at least ten biomarkers, at least eleven biomarkers, or at least twelve biomarkers selected from the biomarkers in Table 6 (with or without additional biomarkers not listed in Table 6) are provided in the panels of metabolites useful for predicting progression from GDM to T2D in a subject in the methods provided herein. Certain non-limiting exemplary panels may be determined using the decision tree modelling disclosed herein. For example, in an embodiment, the plurality of metabolites, also referred to herein as a “panel”, comprises PCaeC40:5 and three or more of: hexoses, branched chain amino acids (e.g., Leu, Ile, and Val), and SM(OH)C14:1. For example, in an embodiment, the plurality of metabolites, comprises SM(OH)C14:1 and three or more of: PCaeC40:5, hexoses, and branched chain amino acids (e.g., xLeu and valine). In an embodiment, the plurality of metabolites further comprises one or more of the metabolites recited in Table 3 and/or Table 4. In an embodiment, the biological sample is a plasma sample obtained from a fasting subject who is 6-9 weeks post-partum.

The biomarkers identified herein provide a number of choices for subsets or panels of biomarkers that can be used to effectively identify progressive GDM. Selection of the appropriate number of such biomarkers may depend on the specific combination of biomarkers chosen. In addition, in any of the methods described herein, except where explicitly indicated, a panel of biomarkers may comprise additional biomarkers not shown in Table 3, 4 or 6.

The method provided herein further involves comparing the levels of the plurality of metabolites in the sample to a corresponding plurality of reference levels in order to predict progression of GDM T2D in the subject. The reference levels may be values that are indicative of a subject who is likely to progress from GDM to T2D or indicative of a subject who is not likely to progress from GDM to T2D, such as, for example, the reference levels set forth in columns 3 (Non-T2D) and 4 (Incident T2D) of Table 4. The determined levels of the plurality of metabolites in a sample may be referred to as a “metabolic signature” of that sample. If such a signature is similar to a signature of a sample obtained from a patient whose GDM progressed to T2D, that signature may be referred to as an “incident T2D metabolic signature”. For example, the inventors found that subjects whose GDM progressed to T2D had a decrease in SMC20:2, SMC18:1, SMC24:1, and glycine, and an increase in hexoses, tyrosine, tryptophan, 2-aminoadipic acid, leucine, isoleucine, valine and AC3 in their fasting plasma samples obtained 6-9 weeks post-partum, relative to subjects whose GDM did not progress to T2D. This is one example of an incident T2D metabolic signature.

The methods provided herein comprise detecting four or more biomarker levels corresponding to four or more biomarkers that are present in the circulation of an individual, such as in serum or plasma, by any number of analytical methods, including any of the analytical methods described herein, such as, for example, mass spectrometry (MS) based assays. For example, one or more of the plurality of metabolites may be assayed and detected directly. For example, one or more of the plurality of metabolites may be assayed and detected indirectly. For example, one or more of the plurality of metabolites may be assayed and detected by detecting a modified version of the assayed metabolite (e.g., detection of a derivative of a metabolite, such as a fatty acid, as described herein).

The biomarkers provided herein are, for example, present at different levels in individuals with progressive GDM as compared to individuals with GDM that does not progress to T2D. Detection of the differential levels of a biomarker in an individual may be used, for example, to permit the determination of whether the individual will develop T2D (i.e., incident T2D). In some embodiments, any of the biomarker panels described herein may be used to monitor the determination of whether GDM is likely to progress to T2D.

In the case of biomarkers whose levels are higher in progressive GDM, in some embodiments, an increase in the level of one or more of the biomarkers during the course of follow-up treatment may be indicative of GDM to T2D progression, whereas a decrease in the level may indicate that the individual's GDM is moving away from development of T2D. Similarly, in some embodiments, for biomarkers whose levels are lower in progressive GDM, in some embodiments, a decrease in the level of one or more of the biomarkers during the course of follow-up treatment may be indicative of GDM to T2D progression, whereas an increase in the level may indicate that the individual's GDM is moving away from development of T2D. Furthermore, a differential expression level of one or more of the biomarkers in an individual over time may be indicative of the individual's response to a particular therapeutic regimen. In some embodiments, changes in expression of one or more of the biomarkers during follow-up monitoring may indicate that a particular therapy is effective or may suggest that the therapeutic regimen should be altered in some way, such as by changing one or more therapeutic agents and/or dosages or lifestyle regimens.

In some embodiments, the method provided herein may be used in conjunction with other T2D screening methods, such as glucose tests (e.g., fasting glucose and/or 2-hour post-load glucose (2hPG)). For example, the biomarkers may facilitate the medical and economic justification for implementing more aggressive treatments for T2D, more frequent follow-up screening, etc. The biomarkers may also be used to begin treatment in GDM individuals at risk of T2D, but who have not been diagnosed with T2D, if the diagnostic test indicates they are likely to develop T2D.

Methods of Treatment

In some embodiments, following a determination that a subject having GDM is likely to progress to T2D, the subject is treated to prevent or slow progression to T2D.

Treatments to prevent or slow the progression of T2D are known in the art, including but are not limited to, one or more treatments for T2D. Treatments for T2D include, but are not limited to, treatment comprising one or more of: diet regimens, exercise regimens, blood sugar monitoring, insulin therapy (e.g., insulin glulisine (Apidra™), insulin lispro (Humalog™), insulin aspart (Novolog™), insulin glargine (Lantus™), insulin detemir (Levemir™), insulin isophane (Humulin™ N, Novolin™ N)), and medications, such as, but not limited to: metformin, sulfonylureas, meglitinides, thiazolidinediones, DPP-4 inhibitors, GLP-1 receptor agonists, SGLT2 inhibitors. Parameters of diet regimens suitable for preventing or slowing the progression of T2D and for treating T2D that are known in the art may be suitable for use with the method provided herein. Parameters of exercise regimens suitable for preventing or slowing the progression of T2D and for treating T2D that are known in the art may be suitable for use with the method provided herein. Regimens for insulin therapy suitable for preventing or slowing the progression of T2D and for treating T2D that are known in the art may be suitable for use with the method provided herein. Regimens for administration of medications suitable for preventing or slowing the progression of T2D and for treating T2D that are known in the art may be suitable for use with the method provided herein.

In some embodiments, methods of monitoring GDM are provided. In some embodiments, the method of predicting progression of GDM to T2D in a subject, as provided herein is carried out at a first time point, “time 0”. In some embodiments, the method is carried out again at a second time point, “time 1”, which is later than time 0, and optionally, at a third time point, “time 2”, which is later than time 1, and optionally, at a fourth time point, “time 3”, which is later than time 2, etc., in order to monitor the progression of GDM; or to monitor the effectiveness of one or more treatments to prevent or slow the progression of GDM to T2D.

Kits for Use in the Methods Provided Herein

The present disclosure contemplates kits for carrying out the methods provided herein. Such kits typically comprise two or more components required for analysing a plurality of metabolites as disclosed herein. Components of the kit include, but are not limited to, one or more of compounds, reagents, containers, equipment and instructions for using the kit. Accordingly, the methods described herein may be performed by utilizing pre-packaged kits provided herein.

In an embodiment, a kit for use in predicting progression of GDM to T2D in vitro is provided. The kit comprises one or more reagent for derivatization, extraction or extraction of one or more metabolites. In some embodiments, instructions for use of the kit to predict progression of GDM to T2D in vitro are provided. The instructions may comprise one or more protocols for: extracting samples, derivatizing samples, running samples on an analytic instrument (e.g., GC-MS or LC/MS/MS), or detecting metabolites.

The kit may further include materials useful for conducting the present method such as, for example, consumables and the like.

Computer Readable Medium

In an aspect, a computer readable medium having computer executable instructions for evaluating a subject's risk for progressing from GDM to T2D is provided. The computer readable medium comprises: a routine, stored on the computer readable medium and adapted to be executed by a processor, to store metabolite measurement data representing measurements of a plurality of metabolites, including four or more metabolites set forth in Table 3, 4, and/or 6 (e.g., PCaeC40:5, hexoses, Leu, Ile, Val, and SM(OH)C14:1); and a routine stored on the computer readable medium and adapted to be executed by a processor to analyze the metabolite measurement data of the subject to evaluate a risk for progressing from GDM to T2D.

For example, a tangible, non-transitory computer-readable medium (i.e., a medium which does not comprise only a transitory propagating signal per se) comprising the computer-executable instructions associated with the disclosed method(s), such as a local or remote hard disk or hard drive (of any type, including electromechanical magnetic disks and solid-state disks), a memory chip, including, e.g., random-access memory (RAM) and/or read-only memory (ROM), cache(s), buffer(s), flash memory, optical memory such as CD(s) and DVD(s), floppy disks, and any other form of storage medium in or on which information may be stored in a volatile or non-volatile manner, for any duration, included permanently or for brief instances, is provided herein. Such computer-executable instructions, if executed by a computer or machine (e.g., a processor based-system, such as a computer housing a processor), cause the processor, and/or the computer or machine, to perform any of the methods described herein, including those which include the steps of analyzing a biological sample to determine levels of a plurality of metabolites in the sample, comparing the determined levels of the plurality of metabolites in the sample to a corresponding plurality of reference levels in order to predict progression of GDM to T2D in a subject. The functions or method steps may be implemented in a variety of programming languages, and such code or computer readable or executable instructions may be stored or adapted for storage in one or more machine-readable media, such as described above, which may be accessed by a processor-based system to execute the stored code or computer readable or executable instructions.

Medical Diagnostic Test System

In an aspect, a medical diagnostic test system for a subject's risk for progressing from GDM to T2D is provided. The system comprises: a data collection tool adapted to collect metabolite measurement data representative of measurements of a plurality of metabolites in a biological sample from the subject, wherein the plurality of metabolites comprises at least four metabolites set forth in Tables 3, 4, and/or 6 (e.g., PCaeC40:5, hexoses, Leu, Ile, Val, and SM(OH)C14:1); an analysis tool comprising a statistical analysis engine adapted to generate a representation of a correlation between a progression from GDM to T2D and measurements of the plurality of metabolites, wherein the representation of the correlation is adapted to be executed to generate a result; and an index computation tool adapted to analyze the result to determine the individual's risk for progressing from GDM to T2D and represent the result as an index value.

In an embodiment, the system comprises a database containing features of biomarkers characteristic of progressive GDM. The biomarker data (or biomarker information) may be utilized as an input to the computer for use as part of a computer implemented method. The biomarker data includes metabolite measurement data representing measurements of a plurality of metabolites, including four or more metabolites set forth in Table 3, 4, and/or 6, as described herein.

At least some embodiments of the methods and/or systems described herein can be implemented with the use of a computer. For example, the steps of the claimed method may be operational with general purpose or special purpose computing system environments or configurations. Examples of well-known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

Non-limiting embodiments are described by reference to the following Examples, which are not to be construed as limiting.

### Example 1. Research Design and Methods

Study Design:

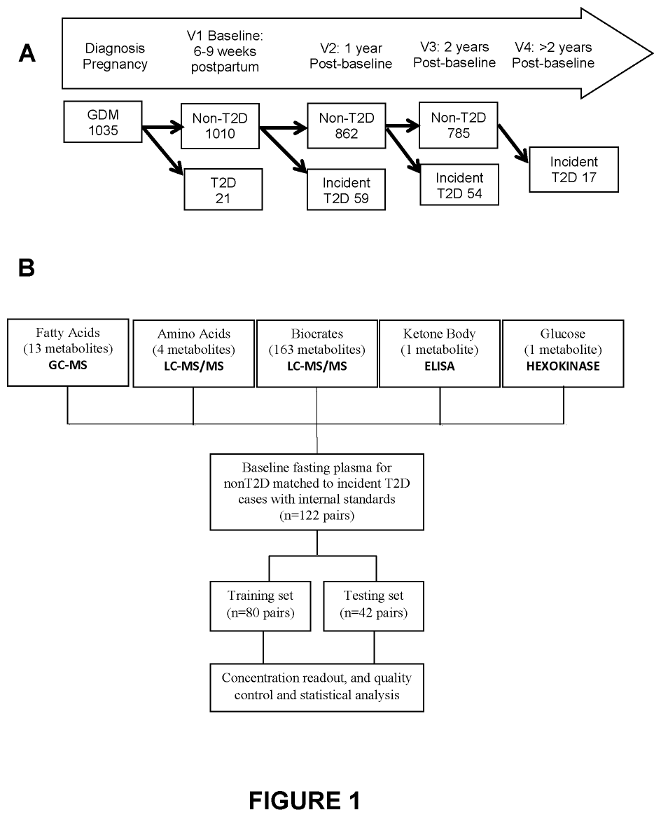

The study design is illustrated in FIG. 1a. At baseline (V1), 21 women with T2D and 4 ineligibles were excluded from the follow up. The study followed 1,010 participants without diabetes who were re-screened annually via OGTTs with retention rates of 85% and 83% for 1 and 2 years, respectively (95% retention overall up to 2 years). Prospective cohort sample sizes for non-T2D and incident T2D shown: 59 developed T2D at 1 years and 54 developed T2D at 2 years, and another 17 women developed T2D beyond 2 to 4 years post-baseline.

The Study of Women, Infant Feeding, and Type 2 diabetes mellitus after GDM Pregnancy (SWIFT) is prospective cohort study that enrolled 1035 racially and ethnically diverse women (aged 20-45 years) who were diagnosed with GDM via a 3-hr 100 g OGTT based on Carpenter and Coustan criteria, had no prior history of diabetes or other serious health conditions, received prenatal care and delivered singleton pregnancies of 35 weeks gestation or longer at a Kaiser Permanente Northern California (KPNC) hospital during 2008-2011. Details of the study recruitment, selection criteria, methodologies and baseline characteristics of the cohort (75% minority women; Asian, Hispanic, and Black, and 25% low-income) have been described previously (24; 25). The SWIFT Study participants provided written consent to attend three in-person study visits at baseline (6-9 weeks post-partum), 1 year and 2 years post-partum that included 2-hr 75 g OGTT and assessments of lactation, intensity and duration, socio-demographics, medical and reproductive history, lifestyle behaviors and anthropometry (24). At each study visit, trained research staff collected and processed plasma samples at the fasting and 2-hr time points during the 75 g OGTT and completed assessments. These plasma samples were analyzed within several weeks for glucose, insulin and subsequently for selected lipids and lipoproteins, as previously described (25; 26). The study design and all procedures were approved by the KPNC Institutional Review Board for the protection of human subjects. Of 1,010 women without T2D at baseline, 959 (95%) had follow-up assessments for T2D status within two years after baseline via annual study OGTTs and electronic medical records to capture diagnoses of diabetes from KPNC clinical laboratory tests within and beyond the 2 years post-baseline (27). T2D diagnosis was based on ADA criteria (24).

Design of Experiment:

Of the 130 incident T2D cases, 113 developed within 2 years post-baseline (27), and another 17 beyond 2 years as of December 2014. Using a nested case-control study design within the prospective cohort, 122 cases (105 within 2 years, and 17 beyond 2 years post-baseline) were matched to non-T2D controls in a 1:1 ratio based on age, pre-pregnancy BMI and race/ethnicity. Age, pre-pregnancy BMI, and ethnicity/race distribution in these excluded cases were not significantly different from cases included in the analysis. The 122 incident T2D cases were split in a 2:1 ratio for the training and testing sets. Importantly, the training set cases were all time-matched to incidence within 2 years, and were used to develop a metabolic risk signature. Subsequently, the testing set, comprising 28 cases within 2 years as well as 14 cases beyond 2 years, was used to independently ensure generalizability of the model.

Metabolite Assay Development:

To assay all metabolites of interest, a total of 182 metabolites were subpanelled into 4 major methods and evaluated in fasting plasma samples collected at 6-9 weeks post-partum (FIG. 1b). The subpanel of 13 free fatty acids and 4 amino acids were selected based on a literature review of over a dozen of T2D metabolomics studies (13-22; 28; 29). These metabolites were chosen on the basis of consistency in trend direction and significance in a minimum of two studies. Both free fatty acid and amino acid subpanel assays were developed in-house as described below in the following relevant sections. In addition, a total of 163 metabolites were assayed using the p150 AbsolutelDQ™ plate technology according to the manufacturer's instructions (Biocrates Life Sciences AG, Austria). All assays were performed by the Analytical Facility for Bioactive Molecules (The Hospital for Sick Children, Toronto, Canada). Beta-hydroxybutyrate (BHB 700190; Cayman Chemicals, USA) was assayed by ELISA while Fasting (FPG) and 2-hour OGTT post-load glucose (2hPG) were assayed as previously described (25). Only metabolites with a coefficient of variation (CV) of <20% for each batch were accepted for the multiplex methods, although the majority had CV of <15%. In addition, values were only accepted if the read concentration was within the dynamic range of the assay.

Amino Acid Analysis:

For amino acid analyses, aliquots (10 μL) of plasma samples and standard mix samples (0.05-50 μg/mL Leu and Ile, 0.005-5 μg/mL AAA and PAG) were spiked with the internal standard mixture (5 μg/mL Leu-d10 and Glu-d3, 0.5 μg/mL PAG-d5 in H₂O+0.1% FA) and extracted by protein precipitation using 600 μL methanol. Samples were then derivatized with 100 μL 3N HCL in n-butanol, evaporated, and reconstituted in 500 μL of the LC/MS/MS mobile phase. LC-MS/MS analysis was performed on an Agilent 1290 HPLC with a Q-Trap 5500 mass spectrometer (AB Sciex). Chromatography was performed isocratically on a Kinetex HILIC column (2.6 μm 100 Å, 50×4.6 mm) (Phenomenex) at a flow rate of 500 μL/min using 5 mM ammonium formate (pH 3.2) in 10/90 water/acetonitrile as the mobile phase. Data was acquired by scheduled MRM.

Free Fatty Acids Analysis: For selected fatty acids, aliquots (20 μL) of plasma samples and standard mix samples [(palmitic (C16:0), palmitoleic (C16:1 n-7), cis-7-hexadecenoic (C16:1 n-9), stearic (C18:0), oleic (C18:1 n-9), vaccenic (C18:1 n-7), linoleic (C18:2), α-linolenic (C18:3), arachidic (C20:0), eicosenoic (C20:1 n-7), arachidonic (C20:4), eicosapentaenoic (EPA; C20:5), docosapentaenoic (DPA; C22:5), and docosahexaenoic (DHA; C22:6) acids)] were spiked with internal standards [(myristic acid-d3 (C14:0-d3), palmitoleic acid-d14 (C16:1-d14), heptadecanoic acid (C17:0) and eicosanoic acid-d3 (C20:0-d3))]. Samples were then acidified with 1 M HCl, and extracted twice with 1 mL of hexane. The combined hexane phases were taken to dryness and derivatized with equal amounts of 1% pentafluorobenzyl bromide and 1% diisopropylamine, evaporated, and reconstituted in 200 μL of hexane. The samples were then injected on the GC-MS system. Excellent separation on the chromatograph was observed for every fatty acid, except for oleate and vaccenate. These two were thus combined to give a total concentration for C18:1

Statistical analysis: Testing and training set characteristics at baseline were compared using chi-squared statistics for categorical variables (race, education, perinatal characteristics, medication use) and by comparison of means for continuous variables using analysis of variance (fasting plasma lipids and glucose, age, BMI). A two-tailed independent t-test was computed to determine significant differences between non-T2D and incident T2D in the baseline metabolite concentrations, with alpha value set at p<0.05 using SPSS Statistics version 20 (SPSS Inc. IBM: USA) and then p-values corrected for multiple comparisons with the Benjamini-Hochberg method using RStudio software version 0.99.486 (Boston, Mass., USA). Predictive modelling was performed using WEKA (University of Waikato, New Zealand). The best model was selected as the one with the highest score in the summation of the discriminative power from the receiver operating curves (ROC) and the F-score (30), a measure that places greater weight on detecting future cases. The J48 machine learner was optimized to develop a broad classifier by setting the confidence threshold to 0.5 and the minimum object in the leaf node to 14. The Naïve Bayes classifier was used as the default parameter setting in the WEKA software. Sensitivity, specificity and precision were further calculated from the classification plot for both the training and testing set.

Pearson's correlation coefficients were calculated to analyze the relationship between significant metabolites and baseline clinically-relevant parameters baseline BMI, FPG, 2-hPG, fasting insulin, and HOMA-IR) using SAS for Windows (9.1.3, SAS Institute Inc., Cary, N.C., USA).

### Example 2. Results

Baseline sociodemographic and clinical characteristics of training and testing sets are summarized in Table 2. While the mean age of women in the training set was significantly younger (p<0.05) compared to testing set, no statistically significant differences in any other baseline or prenatal clinical characteristics were found. The race/ethnicity distribution in both training and testing sets were similar. There was no statistically significant difference in either pre-pregnancy or baseline (6-9 weeks post-partum) BMI, total caloric intake or physical activity. A greater proportion of T2D incident cases had a family history of T2D in the testing set compared to the training set. At baseline, there were statistically significant higher mean FPG, 2-hPG, hPG, fasting insulin and a higher proportion treated with insulin or oral diabetes medications during pregnancy among incident T2D compared to non-T2D (p<0.05) in both sets. Mean HOMA-IR was higher for T2D versus non-T2D (p<0.05) only in the training set.

TABLE 2
Baseline (6-9 weeks post-partum) and follow-up characteristics of SWIFT
women with GDM in the training and testing set (n = 122 pairs). Date presented are Mean
(SD) unless otherwise noted or n (%). Plasma values are from the SWIFT database (25).

| | Training Set | | | | Testing Set | | | |
|---|---|---|---|---|---|---|---|---|
| | | | Incident | | | | Incident | |
| | Non-T2D | | T2D | | Non-T2D | | T2D | |
| Characteristics | (n = 80) | | (n = 80) | | (n = 42) | | (n = 42) | |
| Sociodemographic/Clinical | | | | | | | | |
| Age, years | 33.1 | (4.5) | 33.3 | (5.2) | 35.1 | (5.5)† | 35.4 | (5.5)† |
| Race/Ethnicity, n | | | | | | | | |
| Non-Hispanic White | 13 | (16) | 12 | (15) | 8 | (19) | 9 | (21) |
| Asian, (East, South, Southeast) | 26 | (33) | 26 | (33) | 13 | (31) | 10 | (24) |
| Non-Hispanic Black | 10 | (12) | 10 | (12) | 2 | (5) | 5 | (12) |
| Hispanic | 31 | (39) | 31 | (39) | 17 | (41) | 17 | (41) |
| Other | 0 | (0) | 1 | (1) | 2 | (5) | 1 | (2) |
| Parity, n | | | | | | | | |
| Primiparous (1 birth) | 31 | (39) | 26 | (33) | 13 | (31) | 16 | (38) |
| Biparous (2 births) | 27 | (34) | 29 | (36) | 14 | (33) | 16 | (38) |
| Multiparous (>2 births) | 22 | (27) | 25 | (31) | 15 | (36) | 10 | (24) |
| GDM prenatal treatment, n | | | Chi-sq * | | | | Chi-sq * | |
| Diet Only | 50 | (63) | 33 | (41) | 29 | (69) | 19 | (45) |
| Oral Medications | 28 | (35) | 38 | (48) | 13 | (31) | 17 | (40) |
| Insulin | 2 | (2) | 9 | (11) | 0 | (0) | 6 | (14) |
| Gestational Age at GDM diagnosis | 24.4 | (7.5) | 22.0 | (8.6) | 25.0 | (7.1) | 23.3 | (8.1) |
| (wks) | | | | | | | | |
| Pre-pregnancy BMI, kg/m² | 33.3 | (8.3) | 33.5 | (8.4) | 32.6 | (7.5) | 33.1 | (7.6) |
| Postpartum 6-9 weeks BMI, kg/m² | 33.2 | (7.8) | 33.5 | (7.7) | 32.4 | (6.6) | 33.3 | (7.6) |
| Hypertension history, n | 16 | (20) | 19 | (24) | 8 | (19) | 8 | (19) |
| Family history of diabetes, n | 42 | (53) | 45 | (56) | 19 | (33) | 27 | (64)* |
| 6-9 weeks Postpartum, Lifestyle | | | | | | | | |
| Smoker, n | 2 | (3) | 4 | (5) | 1 | (2) | 1 | (2) |
| Physical activity, met-hrs/week | 47.4 | (21.0) | 54.2 | (25.1) | 49.4 | (21.6) | 48.8 | (24.9) |
| Total Energy intake, Kcal/day | 811 | (319) | 805 | (338) | 774 | (340) | 900.4 | (297) |
| Lactation Intensity Groups, n | | | | | | | | |
| Exclusive lactation | 20 | (25) | 10 | (12) | 8 | (19) | 8 | (19) |
| Mostly lactation | 30 | (38) | 28 | (35) | 15 | (36) | 17 | (41) |
| Mostly formula/Mixed | 18 | (22) | 19 | (24) | 10 | (24) | 12 | (29) |
| Exclusive formula | 12 | (15) | 23 | (29) | 9 | (21) | 5 | (12) |
| 6-9 weeks Postpartum, Plasma | | | | | | | | |
| Fasting glucose (FPG), mg/dl | 95 | (8.4) | 103 | (10.5)* | 93.5 | (7.8) | 101.4 | (11.3)* |
| 2-hr Post 75 g OGTT (2hPG), mg/dl | 109 | (25.9) | 132 | (29.5)* | 116 | (28.5) | 132 | (30.2)* |
| Fasting insulin, μU/ml | 26 | (14.8) | 33 | (17.7)* | 25.6 | (12.1) | 29.1 | (20) |
| Fasting triglycerides, mg/dl | 128 | (90.7) | 150 | (105.2) | 134 | (79.6) | 151.3 | (106) |
| Fasting HDL-C, mg/dl | 49 | (13.2) | 49 | (13.0) | 51.5 | (13.0) | 49.4 | (10.9) |
| HOMA-IR | 6.1 | (3.7) | 8.6 | (5.0)* | 5.97 | (3.0) | 7.47 | (5.9) |
| HOMA-B | 299 | (183) | 305 | (156) | 313 | (153) | 284 | (193) |
| Post-baseline, 2-Year Follow Up | | | | | | | | |
| Subsequent Birth, n | 5 | (6) | 5 | (6) | 9 | (21) | 2 | (5)* |
| Follow up in months, median (IQR) | 22.4 | (1.9) | 16.4 | (11.6)* | 21.8 | (2.8) | 18.3 | (12.5) |

*p < 0.05 between incident T2D and non-T2D groups, and
†p < 0.05 between training and testing sets.

TABLE 3
Mean and standard deviation of non-T2D and incident T2D cases,
uncorrected p-value (2-tailed t-test) and corrected p-values for multiple
comparisons with the Benjamini-Hochberg method of the 110 metabolites that
passed all quality control tests (n = 80 pairs, training set) and concentrations
given in μM, except for hexoses, which is provided in mM.

| | | | | | | Un- | |
|---|---|---|---|---|---|---|---|
| | | Non-T2D | | Incident T2D | | corrected | Corrected |
| No | Metabolites | Mean ± SD | | Mean ± SD | | P-value | P-value |
| 1 | 2-Aminoadipic acid | 1.06 | ± 0.44 | 1.27 | ± 0.54 | 8.02E−03 | 1.01E−01 |
| 2 | Arg | 99.72 | ± 21.95 | 105.52 | ± 18.95 | 7.57E−02 | 2.99E−01 |
| 3 | Gln | 511.29 | ± 95.68 | 514.28 | ± 105.81 | 8.52E−01 | 9.19E−01 |
| 4 | Gly | 311.1 | ± 112.63 | 279.14 | ± 71.7 | 3.38E−02 | 2.31E−01 |
| 5 | His | 81.54 | ± 9.5 | 83.85 | ± 12.61 | 1.93E−01 | 4.70E−01 |
| 6 | Ile | 46.94 | ± 9.09 | 51.39 | ± 11.8 | 8.30E−03 | 1.01E−01 |
| 7 | Leu | 115.05 | ± 21.79 | 126.34 | ± 29.01 | 6.05E−03 | 9.50E−02 |
| 8 | Met | 31.16 | ± 5.2 | 32.25 | ± 5.25 | 1.88E−01 | 4.70E−01 |
| 9 | Orn | 73.75 | ± 31.53 | 75.46 | ± 18.29 | 6.76E−01 | 8.53E−01 |
| 10 | Phe | 58.91 | ± 8.29 | 60.67 | ± 9.14 | 2.04E−01 | 4.70E−01 |
| 11 | PAG | 2.2 | ± 1.17 | 2.04 | ± 1.19 | 3.96E−01 | 6.41E−01 |
| 12 | Pro | 187.58 | ± 50.64 | 190.17 | ± 50 | 7.45E−01 | 8.53E−01 |
| 13 | Ser | 117.55 | ± 24.69 | 114.93 | ± 20.52 | 4.66E−01 | 6.83E−01 |
| 14 | Thr | 141.13 | ± 27.78 | 154.77 | ± 43.81 | 1.99E−02 | 1.83E−01 |
| 15 | Trp | 66.76 | ± 8.31 | 70.52 | ± 10.99 | 1.57E−02 | 1.57E−01 |
| 16 | Tyr | 94.82 | ± 17.48 | 106.33 | ± 24.51 | 7.95E−04 | 2.23E−02 |
| 17 | Val | 230.79 | ± 35.52 | 252.44 | ± 45.63 | 1.01E−03 | 2.23E−02 |
| 18 | xLeu⁺ | 200.69 | ± 29.18 | 220.64 | ± 43.67 | 8.63E−04 | 2.23E−02 |
| 19 | Hexoses | 4.7 | ± 0.51 | 5.16 | ± 0.63 | 1.13E−06 | 1.24E−04 |
| 20 | SM (OH) C14:1 | 5.4 | ± 1.24 | 5.06 | ± 1.55 | 1.29E−01 | 4.29E−01 |
| 21 | SM (OH) C16:1 | 2.87 | ± 0.69 | 2.62 | ± 0.8 | 3.87E−02 | 2.31E−01 |
| 22 | SM (OH) C22:1 | 9.9 | ± 2.23 | 9.67 | ± 2.62 | 5.62E−01 | 7.63E−01 |
| 23 | SM (OH) C22:2 | 7.13 | ± 1.45 | 6.59 | ± 1.83 | 3.90E−02 | 2.31E−01 |
| 24 | SM (OH) C24:1 | 0.94 | ± 0.24 | 0.9 | ± 0.26 | 3.25E−01 | 5.68E−01 |
| 25 | SM C16:0 | 83.93 | ± 12.88 | 79.38 | ± 17.72 | 6.50E−02 | 2.83E−01 |
| 26 | SM C16:1 | 13.54 | ± 1.9 | 12.89 | ± 3.06 | 1.03E−01 | 3.55E−01 |
| 27 | SM C18:0 | 17.21 | ± 3.83 | 15.82 | ± 4.19 | 2.98E−02 | 2.31E−01 |
| 28 | SM C18:1 | 8.91 | ± 2.01 | 7.94 | ± 2.21 | 4.11E−03 | 7.54E−02 |
| 29 | SM C20:2 | 0.42 | ± 0.12 | 0.34 | ± 0.12 | 1.33E−04 | 7.33E−03 |
| 30 | SM C24:0 | 14.51 | ± 3.43 | 14.12 | ± 3.6 | 4.92E−01 | 7.01E−01 |
| 31 | SM C24:1 | 26.86 | ± 5.52 | 24.52 | ± 6.44 | 1.47E−02 | 1.57E−01 |
| 32 | LPC a C16:0 | 168.25 | ± 32.25 | 171.54 | ± 36.35 | 5.45E−01 | 7.50E−01 |
| 33 | LPC a C16:1 | 4.44 | ± 1.11 | 4.24 | ± 1.03 | 2.23E−01 | 4.77E−01 |
| 34 | LPC a C17:0 | 3.54 | ± 0.98 | 3.33 | ± 0.96 | 1.77E−01 | 4.70E−01 |
| 35 | LPC a C18:0 | 64.1 | ± 14.75 | 66.94 | ± 16.2 | 2.49E−01 | 4.89E−01 |
| 36 | LPC a C18:1 | 30.48 | ± 7.99 | 29.18 | ± 7.05 | 2.76E−01 | 4.97E−01 |
| 37 | LPC a C18:2 | 38.64 | ± 10.79 | 39.22 | ± 11.58 | 7.45E−01 | 8.53E−01 |
| 38 | LPC a C20:3 | 3.78 | ± 1.18 | 4.01 | ± 1.25 | 2.40E−01 | 4.89E−01 |
| 39 | LPC a C20:4 | 11.81 | ± 3.11 | 11.92 | ± 3.98 | 8.42E−01 | 9.17E−01 |
| 40 | PC aa C28:1 | 3.46 | ± 0.78 | 3.41 | ± 1 | 7.28E−01 | 8.53E−01 |
| 41 | PC aa C30:0 | 3.7 | ± 1.16 | 3.96 | ± 1.25 | 1.78E−01 | 4.70E−01 |
| 42 | PC aa C32:0 | 13.7 | ± 2.94 | 13.69 | ± 3.11 | 9.79E−01 | 9.88E−01 |
| 43 | PC aa C32:1 | 13.04 | ± 6.37 | 14.35 | ± 6.69 | 2.07E−01 | 4.70E−01 |
| 44 | PC aa C32:2 | 5.05 | ± 1.9 | 5.42 | ± 1.92 | 2.30E−01 | 4.77E−01 |
| 45 | PC aa C34:1 | 149.54 | ± 31.4 | 150.14 | ± 30.52 | 9.03E−01 | 9.55E−01 |
| 46 | PC aa C34:2 | 271.39 | ± 32.99 | 277.49 | ± 30.96 | 2.30E−01 | 4.77E−01 |
| 47 | PC aa C34:3 | 17.29 | ± 4.55 | 17.04 | ± 4.14 | 7.15E−01 | 8.53E−01 |
| 48 | PC aa C34:4 | 1.97 | ± 0.6 | 2.1 | ± 0.64 | 1.90E−01 | 4.70E−01 |
| 49 | PC aa C36:1 | 43.92 | ± 12.14 | 45.17 | ± 11.09 | 4.97E−01 | 7.01E−01 |
| 50 | PC aa C36:2 | 200.48 | ± 29.03 | 206.65 | ± 31.14 | 1.97E−01 | 4.70E−01 |
| 51 | PC aa C36:3 | 126.8 | ± 25.99 | 128.65 | ± 25.22 | 6.50E−01 | 8.41E−01 |
| 52 | PC aa C36:4 | 162.87 | ± 30.11 | 164.56 | ± 34 | 7.40E−01 | 8.53E−01 |
| 53 | PC aa C36:5 | 16.1 | ± 5.56 | 17.81 | ± 11.1 | 2.19E−01 | 4.77E−01 |
| 54 | PC aa C36:6 | 0.8 | ± 0.27 | 0.84 | ± 0.45 | 4.22E−01 | 6.55E−01 |
| 55 | PC aa C38:0 | 2.65 | ± 0.73 | 2.65 | ± 0.83 | 9.88E−01 | 9.88E−01 |
| 56 | PC aa C38:3 | 50.18 | ± 14.51 | 53.05 | ± 13.96 | 2.04E−01 | 4.70E−01 |
| 57 | PC aa C38:4 | 101.75 | ± 21.63 | 104.65 | ± 24.8 | 4.32E−01 | 6.55E−01 |
| 58 | PC aa C38:5 | 46.29 | ± 10.53 | 46.41 | ± 12.09 | 9.49E−01 | 9.84E−01 |
| 59 | PC aa C38:6 | 51.86 | ± 19.74 | 50.25 | ± 19.32 | 6.02E−01 | 7.98E−01 |
| 60 | PC aa C40:2 | 0.46 | ± 0.16 | 0.44 | ± 0.12 | 2.06E−01 | 4.70E−01 |
| 61 | PC aa C40:4 | 3.75 | ± 1.23 | 3.99 | ± 1.19 | 2.03E−01 | 4.70E−01 |
| 62 | PC aa C40:5 | 8.91 | ± 2.89 | 9.26 | ± 2.47 | 4.07E−01 | 6.49E−01 |
| 63 | PC aa C40:6 | 18.22 | ± 6.87 | 18.41 | ± 6.93 | 8.64E−01 | 9.23E−01 |
| 64 | PC aa C42:0 | 0.52 | ± 0.11 | 0.5 | ± 0.15 | 2.02E−01 | 4.70E−01 |
| 65 | PC aa C42:6 | 0.46 | ± 0.1 | 0.47 | ± 0.1 | 8.34E−01 | 9.17E−01 |
| 66 | PC ae C32:1 | 2.82 | ± 0.59 | 2.66 | ± 0.64 | 9.93E−02 | 3.52E−01 |
| 67 | PC ae C34:0 | 1.33 | ± 0.38 | 1.32 | ± 0.38 | 8.11E−01 | 9.17E−01 |
| 68 | PC ae C34:1 | 8.85 | ± 1.93 | 8.26 | ± 1.86 | 5.02E−02 | 2.32E−01 |
| 69 | PC ae C34:2 | 13.07 | ± 3.35 | 12.85 | ± 4.03 | 7.10E−01 | 8.53E−01 |
| 70 | PC ae C34:3 | 9.01 | ± 3.14 | 8.45 | ± 3.32 | 2.73E−01 | 4.97E−01 |
| 71 | PC ae C36:1 | 9.96 | ± 2.3 | 9.47 | ± 2.49 | 2.02E−01 | 4.70E−01 |
| 72 | PC ae C36:2 | 15.2 | ± 3.52 | 14.16 | ± 3.89 | 7.80E−02 | 2.99E−01 |
| 73 | PC ae C36:3 | 8.26 | ± 2.35 | 7.97 | ± 2.37 | 4.27E−01 | 6.55E−01 |
| 74 | PC ae C36:4 | 17.94 | ± 4.1 | 17.97 | ± 5.84 | 9.68E−01 | 9.88E−01 |
| 75 | PC ae C36:5 | 10.55 | ± 2.76 | 10.74 | ± 4.24 | 7.30E−01 | 8.53E−01 |
| 76 | PC ae C38:4 | 12.65 | ± 2.62 | 12.04 | ± 3.21 | 1.90E−01 | 4.70E−01 |
| 77 | PC ae C38:5 | 15.34 | ± 3.05 | 15.21 | ± 4.29 | 8.27E−01 | 9.17E−01 |
| 78 | PC ae C38:6 | 6.54 | ± 1.64 | 6.4 | ± 2.06 | 6.40E−01 | 8.38E−01 |
| 79 | PC ae C40:2 | 2.22 | ± 0.49 | 2.06 | ± 0.55 | 5.05E−02 | 2.32E−01 |
| 80 | PC ae C40:4 | 3.34 | ± 0.78 | 3.08 | ± 0.96 | 6.68E−02 | 2.83E−01 |
| 81 | PC ae C40:5 | 4.81 | ± 1.21 | 4.36 | ± 1.59 | 4.32E−02 | 2.31E−01 |
| 82 | PC ae C40:6 | 3.45 | ± 0.78 | 3.31 | ± 1.11 | 3.33E−01 | 5.72E−01 |
| 83 | PC ae C42:5 | 2.27 | ± 0.46 | 2.08 | ± 0.59 | 2.42E−02 | 2.05E−01 |
| 84 | PC ae C44:3 | 0.313 | ± 0.08 | 0.306 | ± 0.08 | 5.89E−01 | 7.90E−01 |
| 85 | PC ae C44:4 | 0.43 | ± 0.09 | 0.4 | ± 0.1 | 8.19E−02 | 3.00E−01 |
| 86 | PC ae C44:5 | 1.18 | ± 0.25 | 1.09 | ± 0.32 | 4.47E−02 | 2.31E−01 |
| 87 | PC ae C44:6 | 1.04 | ± 0.25 | 0.97 | ± 0.31 | 1.53E−01 | 4.70E−01 |
| 88 | AC0 | 37.12 | ± 7.85 | 37.63 | ± 10.17 | 7.22E−01 | 8.53E−01 |
| 89 | AC10 | 0.25 | ± 0.08 | 0.22 | ± 0.06 | 4.63E−02 | 2.31E−01 |
| 90 | AC2 | 5.5 | ± 1.53 | 5.22 | ± 1.6 | 2.62E−01 | 4.91E−01 |
| 91 | AC3 | 0.28 | ± 0.08 | 0.31 | ± 0.1 | 4.55E−02 | 2.31E−01 |
| 92 | AC4 | 0.18 | ± 0.07 | 0.18 | ± 0.06 | 4.75E−01 | 6.88E−01 |
| 93 | AC5 | 0.098 | ± 0.028 | 0.102 | ± 0.030 | 3.09E−01 | 5.47E−01 |
| 94 | AC8:1 | 0.17 | ± 0.07 | 0.16 | ± 0.07 | 8.20E−01 | 9.17E−01 |
| 95 | AC18:1 | 0.090 | ± 0.024 | 0.086 | ± 0.025 | 3.73E−01 | 6.27E−01 |
| 96 | AC18:2 | 0.04 | ± 0.012 | 0.04 | ± 0.012 | 9.82E−01 | 9.88E−01 |
| 97 | Myristic acid | 11.49 | ± 4.21 | 10.66 | ± 4.79 | 2.48E−01 | 4.89E−01 |
| | (C14:0) | | | | | | |
| 98 | Palmitic acid | 203.14 | ± 78 | 197.8 | ± 89.91 | 6.91E−01 | 8.53E−01 |
| | (C16:0) | | | | | | |
| 99 | Hexadecenoic acid | 21.94 | ± 8.78 | 20.48 | ± 11.7 | 3.76E−01 | 6.27E−01 |
| | (C16:1 n-7) | | | | | | |
| 100 | Palmitoleic acid | 2.76 | ± 0.96 | 2.45 | ± 0.86 | 3.86E−02 | 2.31E−01 |
| | (C16:1 n-9) | | | | | | |
| 101 | Stearic aicd (C18:0) | 43.22 | ± 25.11 | 46.52 | ± 36.3 | 5.09E−01 | 7.09E−01 |
| | Oleic Acid & | | | | | | |
| 102 | Vaccenic Acid | 284.9 | ± 143.69 | 264.78 | ± 143.65 | 3.82E−01 | 6.27E−01 |
| | (C18:1 n-9, n-7) | | | | | | |
| 103 | Linoleic acid | 197.52 | ± 77.79 | 183.65 | ± 74.2 | 2.55E−01 | 4.91E−01 |
| | (C18:2) | | | | | | |
| 104 | Alpha-linolenic | 8.56 | ± 3.95 | 8.52 | ± 4.23 | 9.46E−01 | 9.84E−01 |
| | acid (C18:3) | | | | | | |
| 105 | Eicosenoic acid | 1.46 | ± 0.61 | 1.36 | ± 0.55 | 2.63E−01 | 4.91E−01 |
| | (C20:1) | | | | | | |
| 106 | Arachidonic acid | 16.39 | ± 7.82 | 17.02 | ± 14.27 | 7.33E−01 | 8.53E−01 |
| | (C20:4) | | | | | | |
| 107 | Eicosapentaenoic | 1.72 | ± 0.99 | 2.17 | ± 3 | 2.09E−01 | 4.70E−01 |
| | acid (C20:5) | | | | | | |
| 108 | Docosapentaenoic | 0.93 | ± 0.4 | 1 | ± 0.71 | 4.35E−01 | 6.55E−01 |
| | acid (C22:5) | | | | | | |
| 109 | Docosahexaenoic | 3.86 | ± 2.33 | 4.29 | ± 4.71 | 4.63E−01 | 6.83E−01 |
| | acid C22:6) | | | | | | |
| 110 | Beta-hydroxybutyrate | 137.35 | ± 93.18 | 172.76 | ± 151.56 | 7.89E−02 | 2.99E−01 |

⁺Metabolise was assayed using both Biocrates plate technology and in-house method but xleu was excluded for prediction analysis.
AC, acylcarnitines;
Arg, arginine,
Gln, glutamine;
Gly, glycine;
His, histidine;
Ile, isoleucine;
Leu, leucine;
LPC, lysophosphatidylcholine;
PC, phosphatidylcholine;
Met, methionine;
Orn, ornithine;
PAG, phenyl acetyl glutamine;
Phe, phenylalanine;
Pro, proline;
Ser, serine;
SM, sphingolipids;
Thr, threonine;
Try, tryptophan;
Try, tyrosine;
Val, valine;
xleu, xleucine.

A total of 110 metabolites passed all quality control criteria as described above and set forth in Table 3. In the training set, a two-tailed independent t-test was carried out, with 22 metabolites found to significantly differ between T2D and non-T2D (Table 4).

The metabolites 2-aminoadipic acid (p<0.008), Ile (p<0.008), Leu (p<0.006), Thr (p<0.01), Trp (p<0.01), Tyr (p<0.0007), Val (p<0.001), xLeu (p<0.0008), Hexose (p<0.000001) and AC3 (p<0.04) levels were significantly elevated in incident T2D compared to non-T2D (Table 4).

In contrast, metabolites Gly (p<0.03), SM (OH) C16:1 (p<0.03), SM (OH) C22:2 (p<0.03), SM C18:0 (p<0.02), SM C18:1 (p<0.004), SM C20:2 (p<0.001), SM C24:1 (p<0.01), PC ae C40:5 (p<0.04), PC ae C42:5 (p<0.02), PC ae C44:5(p<0.04), AC10 (p<0.04) and free fatty acid palmitoleic acid (C16:1 n9) (p<0.03) were decreased in incident T2D compared to non-T2D (Table 4).

Tyr, Val, xLeu, hexoses and SM C20:2 remained statistically significant after Benjamini-Hochberg correction for multiple comparisons (Table 4).

TABLE 4
Metabolites significantly differ in incident T2D in the training set (n = 80 pairs)
and concentrations given in μM, except for hexoses, which is provided in mM.

| | | Non-T2D | | Incident T2D | | Uncorrected | *Corrected |
|---|---|---|---|---|---|---|---|
| No | Metabolites | Mean ± SD | | Mean ± SD | | P-value | P-value |
| 1 | 2-Aminoadipic acid | 1.06 | ± 0.44 | 1.27 | ± 0.54 | 8.02E−03 | 1.01E−01 |
| 2 | Gly | 311.1 | ± 112.63 | 279.14 | ± 71.7 | 3.38E−02 | 2.31E−01 |
| 3 | Ile | 46.94 | ± 9.09 | 51.39 | ± 11.8 | 8.30E−03 | 1.01E−01 |
| 4 | Leu | 115.05 | ± 21.79 | 126.34 | ± 29.01 | 6.05E−03 | 9.50E−02 |
| 5 | Thr | 141.13 | ± 27.78 | 154.77 | ± 43.81 | 1.99E−02 | 1.83E−01 |
| 6 | Trp | 66.76 | ± 8.31 | 70.52 | ± 10.99 | 1.57E−02 | 1.57E−01 |
| 7 | Tyr | 94.82 | ± 17.48 | 106.33 | ± 24.51 | 7.95E−04 | 2.23E−02 |
| 8 | Val | 230.79 | ± 35.52 | 252.44 | ± 45.63 | 1.01E−03 | 2.23E−02 |
| 9 | xLeu⁺ | 200.69 | ± 29.18 | 220.64 | ± 43.67 | 8.63E−04 | 2.23E−02 |
| 10 | Hexoses | 4.7 | ± 0.51 | 5.16 | ± 0.63 | 1.13E−06 | 1.24E−04 |
| 11 | SM (OH) C16:1 | 2.87 | ± 0.69 | 2.62 | ± 0.8 | 3.87E−02 | 2.31E−01 |
| 12 | SM (OH) C22:2 | 7.13 | ± 1.45 | 6.59 | ± 1.83 | 3.90E−02 | 2.31E−01 |
| 13 | SM C18:0 | 17.21 | ± 3.83 | 15.82 | ± 4.19 | 2.98E−02 | 2.31E−01 |
| 14 | SM C18:1 | 8.91 | ± 2.01 | 7.94 | ± 2.21 | 4.11E−03 | 7.54E−02 |
| 15 | SM C20:2 | 0.42 | ± 0.12 | 0.34 | ± 0.12 | 1.33E−04 | 7.33E−03 |
| 16 | SM C24:1 | 26.86 | ± 5.52 | 24.52 | ± 6.44 | 1.47E−02 | 1.57E−01 |
| 17 | PC ae C40:5 | 4.81 | ± 1.21 | 4.36 | ± 1.59 | 4.32E−02 | 2.31E−01 |
| 18 | PC ae C42:5 | 2.27 | ± 0.46 | 2.08 | ± 0.59 | 2.42E−02 | 2.05E−01 |
| 19 | PC ae C44:5 | 1.18 | ± 0.25 | 1.09 | ± 0.32 | 4.47E−02 | 2.31E−01 |
| 20 | AC10 | 0.25 | ± 0.08 | 0.22 | ± 0.06 | 4.63E−02 | 2.31E−01 |
| 21 | AC3 | 0.28 | ± 0.08 | 0.31 | ± 0.1 | 4.55E−02 | 2.31E−01 |
| 22 | Palmitoleic | 2.76 | ± 0.96 | 2.45 | ± 0.86 | 3.86E−02 | 2.31E−01 |
| | acid (C16:1 n9) | | | | | | |

*p values are corrected for multiple comparisons with the Benjamini-Hochberg method and significant metabolites were highlighted in bold text.
⁺Metabolise was assayed using both Biocrates plate technology and in-house method but xleu was excluded for prediction analysis.
AC, acylcarnitines;
Gly, glycine;
Ile, isoleucine;
Leu, leucine;
Thr, threonine;
Try, tryptophan;
Try, tyrosine;
Val, valine;
xLeu, xleucine;
PC, phosphatidylcholine;
SM, sphingolipids.

To identify a set of metabolites with accurate prediction of future T2D we selected a rigorous method of splitting data into training (model building) and testing (model verification) over methods such as cross validation and holdout. Several methods of attribute selection were explored. First, attributes were ranked by predictive capacity and then trained and tested in a Naïve Bayes model. While this initial model worked well in a 10-fold cross-validation it performed poorly in the testing set, indicating that this method of attribute selection contained dataset specific biases (data not shown). Next, the J48 decision tree method using random sampling of attributes to build trees and then select and prune the trees to identify the best preforming attributes (the metabolite model) was used to create the model. We optimized the J48 model by increasing the confidence threshold to 0.5 and the minimum number of subjects to 14. These settings ensured a broad classifier model not prone to over fitting.

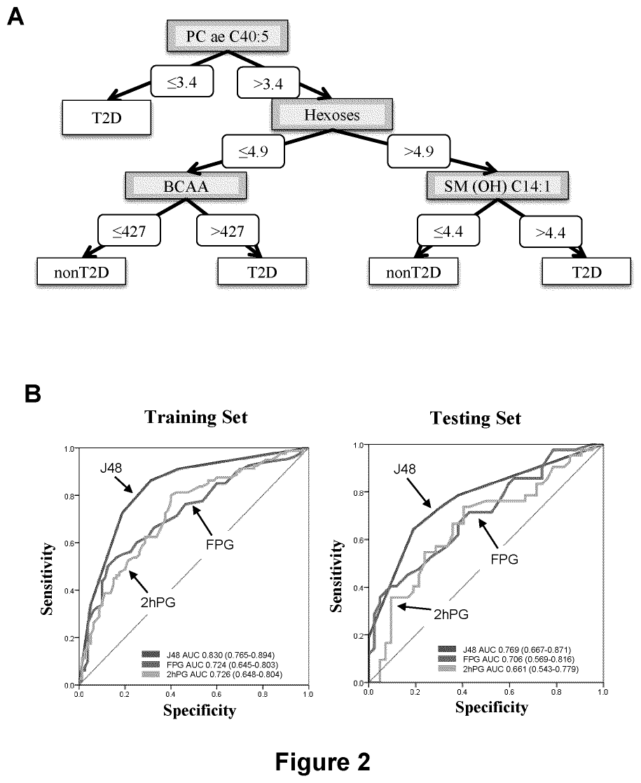

The resulting metabolite model had a high summation of AUC and F-score in the training set (FIG. 2A), relying on only a few metabolites: PC ae C40:5, hexoses, BCAA (Val, Leu, Ile), and SM (OH) C14:1. Baseline (6-9 weeks post-partum) FPG alone predicted T2D incidence in the training set, with an AUC of 0.724 (95% CI, 0.645-0.803, p<0.0001), sensitivity 60.0%, specificity 75.0%, F score 0.649 and total score 1.373. In contrast, the metabolite model resulted in an AUC of 0.830 (95% CI, 0.765-0.894, p<0.000001), with sensitivity 86.3%, specificity 69%, F score 0.793 and total score 1.623. We next applied the metabolite model and the FPG model against the testing data set and assessed relative performance using ROC curves (FIG. 2B). The FPG model was worse at predicting T2D, with AUC 0.706 (95% CI, 0.569-0.816, p<0.01), sensitivity 57.0%, specificity 66.7%, F score 0.6 and total score 1.306. In contrast, the metabolite model performed well with an AUC 0.769 (95% CI, 0.667-0.871, p<0.001), sensitivity 73.8%, specificity 69%, F score 0.721 and total score 1.49 (Table 5). The metabolite model also outperformed the use of 2hPG in both the training set (AUC 0.726, F score 0.6309, total score 1.357) and testing set (AUC 0.661, F-score 0.615, total score 1.276).

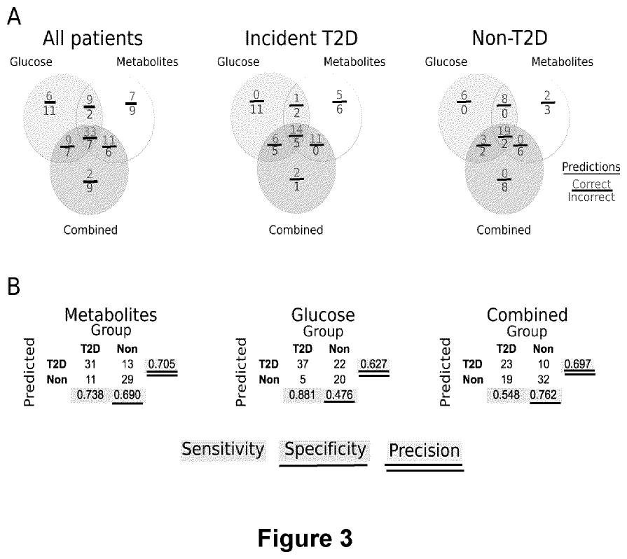

Using FPG and the 2hPG we could build a model using J48 decision tree method (the glucose model). The glucose model had greater sensitivity (Se) but worse precision (P) and specificity (Sp) compared to the metabolite model (glucose model P=0.627, Se=0.881, Sp=0.476; metabolite model: P=0.705, Se=0.738, Sp=0.690). To determine if combining the glucose model and metabolite model (the combined model) could improve prediction we built an optimized Naïve Bayes classifier model combining the four metabolites species and glucose data (FPG and 2hPG). The combined model showed worse prediction compared to metabolites alone (P=0.697, Se=0.548, Sp=0.762). Of the three models, the metabolite only model outperformed the latter two models with the highest AUC and F score (Table 5). The predictions from the three models (metabolite, glucose and combined metabolite-glucose) were directly compared in a Venn diagram to determine the similarities and differences between the models (FIG. 3).

TABLE 5
Comparison of FPG, 2hPG and metabolites optimized machine learning performance, indicating
greatest performance in the metabolite model. Data presented in mean and (95% CI).

| | | Optimized | | | | | | | Best model |
|---|---|---|---|---|---|---|---|---|---|
| | | Machine | | | | | | | Score |
| | | learner | | | | | | | (F score + |
| Sets | Parameters | Algorithm | AUC | Sensitivity | Specificity | Accuracy | Precision | F-score | AUC) |
| Training | FPG | LR | 0.724 | 60.00% | 75.00% | 67.50% | 70.60% | 64.90% | 1.373 |
| | | | (0.645-0.803) | | | | | | |
| | 2hPG | LR | 0.726 | 58.75% | 72.50% | 65.63% | 68.12% | 63.09% | 1.3569 |
| | | | (0.648-0.804) | | | | | | |
| | Metabolite | DT | 0.830 | 86.30% | 68.80% | 77.50% | 73.40% | 79.30% | 1.623 |
| | model | | (0.765-0.894) | | | | | | |
| Testing | FPG | LR | 0.706 | 57.10% | 66.70% | 61.90% | 63.20% | 60.00% | 1.306 |
| | | | (0.596-0.816) | | | | | | |
| | 2hPG Model | LR | 0.661 | 57.10% | 71.40% | 64.30% | 66.70% | 61.50% | 1.276 |
| | | | (0.543-0.779) | | | | | | |
| | Metabolite | DT | 0.769 | 73.80% | 69.10% | 71.40% | 70.50% | 72.10% | 1.490 |
| | model | | (0.667-0.871) | | | | | | |
| | Glucose | DT | 0.732 | 88.10% | 47.60% | 67.90% | 62.70% | 73.30% | 1.465 |
| | model (FPG | | | | | | | | |
| | and 2hPG) | | | | | | | | |
| | Combined | NB | 0.754 | 54.80% | 76.20% | 65.50% | 69.70% | 61.30% | 1.367 |
| | model | | | | | | | | |

LR: Logistic regression, DT: J48 Decision tree NB: Naïve Bayes.

From the comparisons of the three models (FIG. 3) the combined model showed improvement in capturing all 6 future T2D cases solely predicted by the glucose model and missed by the metabolite model (correct predictions underlined in FIG. 3). The glucose model could only capture 11 of 16 future T2D cases predicted by the metabolites model. The combined model fared worse in prediction of controls with 8 unique false negatives (predicted as diabetic; FIG. 3).

Pearson correlation coefficients were calculated between the 22 metabolites that significantly differ between incident T2D and non-T2D in the training set, metabolite selected by machine learning and 5 baseline clinical parameters that significantly differed between incident T2D and non-T2D in both training and testing sets (BMI, fasting glucose, 2-hour post-load glucose, fasting insulin and HOMA-IR). SM C24:1 most significantly and negatively correlated with BMI (p<0.0005, r=−0.277). The correlations of 2-AAA, Ile, AC3, hexoses and SM C20:2 were most significant with fasting glucose level (p<0.0005, r=0.283, 0.278, 0.306, 0.826, and −0.284, respectively). After 2-hr post-load, total hexoses were most significantly correlated with glucose levels (p<0.005, r=0.211) as expected. All other metabolites, with the exception of palmitoleic acid, significantly correlated with both fasting insulin and HOMA-IR (Table 6). Interestingly, among all 22 significant metabolites, glycine and hexoses were the only metabolites to correlate significantly to all 5 clinical parameters; BMI (r=−0.151, 0.160), fasting glucose (r=−0.192, 0.826), 2-hour post-load glucose (r=−0.173, 0.211), fasting insulin (r=−0.279, 0.311) and HOMA-IR (r=−0.281, 0.429). SM (OH) C14:1 correlated negatively with BMI, FPG, 2hPG, fasting insulin and HOMA-IR like other SMs investigated in this study.

TABLE 6
Pearson correlation coefficients (r) between 22 metabolites that
significantly differ in incident T2D compared to non-T2D, as
well as metabolite selected by machine learning (SM (OH)
C14:1), in the training set (80 pairs) at baseline and
clinical parameters BMI, fasting glucose, 2-hour
post-load glucose, fasting insulin and HOMA-IR at baseline.

| | | | 2-hr | | |
|---|---|---|---|---|---|
| | | | Post 75 g | | |
| | | Fasting | OGTT | Fasting | |
| Parameter & | BMI | Glucose | (Glucose | Insulin | HOMA- |
| metabolite | (kg/m²) | mg/dl) | (mg/dl) | (μU/ml) | 1R |
| 2-AAA | 0.210** | 0.283*** | 0.115 | 0.335*** | 0.353*** |
| Gly | −0.151⁺ | −0.192* | −0.173* | −0.279*** | −0.281*** |
| Ile | 0.230** | 0.278*** | 0.144 | 0.415*** | 0.437*** |
| Leu | 0.055 | 0.242** | 0.15* | 0.343*** | 0.367*** |
| Thr | 0.218** | 0.156* | 0.025 | 0.150⁺ | 0.153⁺ |
| Trp | −0.161* | 0.22** | 0.061 | 0.171* | 0.187* |
| Tyr | 0.205** | 0.252** | 0.028 | 0.335*** | 0.353*** |
| Val | 0.073 | 0.235** | 0.161* | 0.409*** | 0.418*** |
| AC10 | −0.022 | −0.165* | 0.139 | −0.201* | −0.202* |
| AC3 | 0.104 | 0.306*** | 0.184* | 0.362*** | 0.387*** |
| xLeu⁺ | 0.118 | 0.311*** | 0.197* | 0.481*** | 0.508*** |
| Hexoses | 0.16* | 0.826*** | 0.211** | 0.311*** | 0.429*** |
| Palmitoleic acid | 0.246** | −0.1 | −0.009 | 0.098 | 0.068 |
| (C16:1n9) | | | | | |
| PC ae C40:5 | −0.252** | −0.054 | 0.081 | −0.329*** | −0.311*** |
| PC ae C42:5 | −0.115 | −0.033 | 0.018 | −0.266*** | −0.252** |
| PC ae C44:5 | −0.006 | −0.177* | −0.182* | −0.204** | −0.217** |
| SM C18:0 | −0.181* | −0.150* | 0.028 | −0.266*** | −0.272*** |
| SM C18:1 | −0.049 | −0.157* | −0.039 | −0.254** | −0.263*** |
| SM C20:2 | −0.092 | −0.284*** | −0.122 | −0.358*** | −0.376*** |
| SM C24:1 | −0.277*** | −0.246** | −0.025 | −0.475*** | −0.475*** |
| SM (OH) C14:1 | −0.136 | −0.207* | −0.175* | −0.257** | −0.279*** |
| SM (OH) C16:1 | −0.161* | −0.199* | −0.087 | −0.315*** | −0.329*** |
| SM (OH) C22:2 | −0.201* | −0.226** | −0.034 | −0.378*** | −0.385*** |

```
  ⁺p = 0.05,
  *p < 0.05,
  **p < 0.005,
***p < 0.0005.
```

### Example 3. Discussion

GDM represents one of the strongest risk factors for the development of T2D, and identifies young women of whom 20-50% may develop T2D within 5 years after delivery (1). Metzger et al. reported that greater severity of hyperglycemia during pregnancy predicted T2D conversion within 6 months post-partum as opposed to 5 years, and that higher pre-pregnancy BMI increased the risk of T2D within 5 years post-partum (31). The Diabetes Prevention Research Group reported a greatly reduced risk of T2D progression among women with a history of GDM by either a lifestyle modification or metformin treatment, with T2D incidence of 10-15% within 10 years compared to 50% in the standard care group (32). Nevertheless many women with GDM hold a false perception of low risk status for future diabetes (8; 9). Thus, diabetes screening is suboptimal during the post-partum period because of the time-consuming glucose tolerance testing and required fasting period.

Herein, we explored a combination of several significantly altered metabolites for prediction of incident T2D compared with clinical parameters, the FPG and 2hPG among women matched on age, race/ethnicity and BMI. Our metabolite model predicts T2D above and beyond the risk contributed by obesity. Several metabolites were statistically significant predictors of incident T2D. Some of these were previously associated with T2D in cross-sectional metabolomics studies, suggesting that GDM women at risk of progressing to T2D present a more “T2D-like metabolite profile” within the very short time frame of 2 months post-partum compared to women who will remain non-diabetic. Women who developed T2D were also more likely to have been treated with insulin or oral medication during pregnancy, underscoring the predictive value of the severity of glucose intolerance during pregnancy.

Comparison of three T2D predictive models identified the metabolite model, disclosed herein, as the most balanced for type-I (false positive) and type-II (false negative) error over the glucose model. A combined model of metabolites and glucose could improve capture of future T2D over glucose alone, but with higher false positive prediction rates than the metabolite model. This increased type-I error suggests a conflict between the predictions arising from the metabolite or glucose models. Alternatively, these false positive predictions of future diabetes may represent detection of individuals that will develop diabetes beyond the two-year window of our current study.

Our study, using machine learning prediction, revealed two novel metabolites as being predictive of incident T2D: PC ae C40:5 and SM (OH) C14:1. Interestingly, PC ae C40:5 was significantly decreased in incident T2D and negatively correlated with BMI, fasting insulin and HOMA-IR. Interestingly, the machine learning-selected metabolite SM (OH) C14:1 was not previously associated with T2D incidence. By “associated”, we mean having a statistical metric indicating a probability that a certain metabolite is associated with T2D incidence. This is because in predictive modeling, in contrast to traditional exploratory research, association is not a requirement for variable inclusion (42). Interestingly, similar to other SMs, SM (OH) C14:1 correlated negatively with BMI, FPG and 2hPG, which may partially explain why the combined model did not outperform the metabolite-only model.

We found that several amino acids (2-AAA, Ile, Leu, Thr, Trp, Tyr, Val) were increased in incident T2D subjects, except for glycine, which was significantly decreased, and is a known predictor of T2D (19). The metabolite 2-AAA has been reported to be increased up to 12 years before T2D onset (28). Interestingly, 2-AAA was elevated in women with incident T2D after a previous GDM pregnancy, and positively correlated with insulin resistance. Herein, we also observed an increase in levels of 2-AAA in incident T2D women. However in a study by Fiehn et al, where levels of 2-AAA were assessed in a cross-sectional study of African American women with T2D, no statistical significance was observed (15). Mechanistically, in murine models treated with 2-AAA decreased FPG and enhanced glucose-stimulated insulin secretion in beta cell models were observed (28). It is still to be determined if a similar response exists in humans.

BCAA levels reportedly correlate with insulin resistance in obese subjects (34). Catabolism of BCAAs plays an important role in T2D and Impaired Fasting Glucose (35). Clinical trials have also demonstrated that BCAAs such as leucine, isoleucine and valine are increased up to 7 years before T2D onset (18). Herein, BCAAs were elevated at 6-9 weeks postpartum among women at highest risk of subsequent progression to T2D. Our results indicate that a metabolic profile including elevated BCAAs precedes the onset of T2D rather than being a consequence of T2D, as previously hypothesized.

In the cohort studied herein, we observed higher levels of the hexoses (all 6 carbon sugars such as glucose, fructose, and mannose) for incident T2D, consistent with reports of others (18). Interestingly, in a T2D metabolomics study, Fiehn et al characterized carbohydrates, and found fructose levels to be significantly elevated in obese women with T2D (15). Unlike glucose, fructose stimulates hepatic lipogenesis which may result to hepatic insulin resistance, a key feature of T2D (36).

We also observed herein an overall reduction of sphingomyelin species (SM) in incident T2D compared to non-T2D. Wang et al. confirmed a decrease in SMC20:2, SM C16:0, SM C16:1, among other SM species (19), and Floegel et al. observed a decrease in SM C16:1 and an inverse association with insulin secretion (21). In these nested-case control studies, the decreases were found up to 7 years before T2D incidence. The metabolic breakdown of SM results in ceramides, which is a known to induce beta cell apoptosis (37; 38). Further research is required to determine whether altered concentrations of ceramides mechanistically contribute to T2D and specifically to levels of SM C20:2, the sphingomyelin species that we found to be most significant in the cohort studied herein.

Anderson et al. investigated the lipidome of postpartum women who were normal, and hyperglycemic (non-GDM) or GDM. They observed that phosphatidylcholine (PC_, lysophosphatidylcholine (LPC), acylcarnitines (AC), and free fatty acids (FFAs) had the strongest correlations (39). Lappas et al. applied lipidomics analysis of plasma collected at 12 weeks post-partum in 104 women with a GDM pregnancy who had normal postpartum glucose tolerance (NGT) and later evaluated T2D again at 8-10 years after delivery (23). A model including age, BMI, pregnancy FPG, postnatal FPG, triacylglycerol and total cholesterol and 3 metabolites (CE 20:4, PE(P-36:2) and PS 38:4). In the study provided herein, palmitoleic acid, AC3, and AC10 were significantly altered with incident T2D. Palmitoleic acid levels have been reported to be positively related to T2D among older adults (40), and AC3 is known to be integral in the pathway of BCAA catabolism (34). In previous studies, the incidence of AC10 is unclear: AC10 has been associated with a graded increase among NGT, IGT and T2D individuals, but others found no significant difference in AC10 for T2D compared to control women (14; 41). In contrast, the study provided herein revealed a decrease in AC10 levels in T2D incident subjects.

Presently, the ADA recommends T2D screening via fasting glucose or the 2-hr 75 g OGTT at 6-12 weeks post-partum and thereafter every 1-3 years for women with a prior GDM diagnosis, and more frequent testing if screening results fall within the pre-diabetes ranges. Our metabolomics signature holds the potential to replace the requirement for frequent OGTTs, Surpassing both the issue of lost follow-up and low screening rates with a single fasting measurement. In addition, the metabolic signature provided herein was comparable and outperformed the 2-hour post-load plasma glucose after the OGTT in predicting future T2D incidence within 2 years. Further, the metabolic signature provided herein provides insight into etiology of the transition to T2D in women with previous GDM.

Although the invention has been described with reference to certain specific embodiments, various modifications thereof will be apparent to those skilled in the art without departing from the purpose and scope of the invention as outlined in the claims appended hereto. Any examples provided herein are included solely for the purpose of illustrating the invention and are not intended to limit the invention in any way. Any drawings provided herein are solely for the purpose of illustrating various aspects of the invention and are not intended to be drawn to scale or to limit the invention in any way.

### REFERENCES CITED HEREIN

1. Kim C, Newton K M, Knopp R H: Gestational diabetes and the incidence of type 2 diabetes: a systematic review. Diabetes Care 2002; 25:1862-1868
2. Ferrara A: Increasing prevalence of gestational diabetes mellitus: a public health perspective. Diabetes Care 2007; 30 Suppl 2:S141-146
3. American Diabetes A: Standards of medical care in diabetes—2014. Diabetes Care 2014; 37 Suppl 1:S14-80
4. Shah B R, Lipscombe L L, Feig D S, Lowe J M: Missed opportunities for type 2 diabetes testing following gestational diabetes: a population-based cohort study. BJOG: an international journal of obstetrics and gynaecology 2011; 118:1484-1490
5. Blatt A J, Nakamoto J M, Kaufman H W: Gaps in diabetes screening during pregnancy and postpartum. Obstetrics and gynecology 2011; 117:61-68
6. Bennett W L, Ennen C S, Carrese J A, Hill-Briggs F, Levine D M, Nicholson W K, Clark J M: Barriers to and facilitators of postpartum follow-up care in women with recent gestational diabetes mellitus: a qualitative study. Journal of women's health 2011; 20:239-245
7. Russell M A, Phipps M G, Olson C L, Welch H G, Carpenter M W: Rates of postpartum glucose testing after gestational diabetes mellitus. Obstetrics and gynecology 2006; 108:1456-1462
8. Jones E J, Roche C C, Appel S J: A review of the health beliefs and lifestyle behaviors of women with previous gestational diabetes. Journal of obstetric, gynecologic, and neonatal nursing: JOGNN/NAACOG 2009; 38:516-526
9. Kim C, McEwen L N, Piette J D, Goewey J, Ferrara A, Walker E A: Risk perception for diabetes among women with histories of gestational diabetes mellitus. Diabetes Care 2007; 30:2281-2286
10. Griffin S J, Little P S, Hales C N, Kinmonth A L, Wareham N J: Diabetes risk score: towards earlier detection of type 2 diabetes in general practice. Diabetes/metabolism research and reviews 2000; 16:164-171
11. Rosella L C, Manuel D G, Burchill C, Stukel T A, team P-D: A population-based risk algorithm for the development of diabetes: development and validation of the Diabetes Population Risk Tool (DPoRT). Journal of epidemiology and community health 2011; 65:613-620
12. Deo R C: Machine Learning in Medicine. Circulation 2015; 132:1920-1930
13. Yi L, Yuan D, Che Z, Liang Y, Zhou Z, Gao H, Wang Y: Plasma fatty acid metabolic profile coupled with uncorrelated linear discriminant analysis to diagnose and biomarker screening of type 2 diabetes and type 2 diabetic coronary heart diseases. Metabolomics 2008; 4:30-38
14. Adams S H, Hoppel C L, Lok K H, Zhao L, Wong S W, Minkler P E, Hwang D H, Newman J W, Garvey W T: Plasma acylcarnitine profiles suggest incomplete long-chain fatty acid beta-oxidation and altered tricarboxylic acid cycle activity in type 2 diabetic African-American women. The Journal of nutrition 2009; 139:1073-1081
15. Fiehn O, Garvey W T, Newman J W, Lok K H, Hoppel C L, Adams S H: Plasma metabolomic profiles reflective of glucose homeostasis in non-diabetic and type 2 diabetic obese African-American women. PloS one 2010; 5:e15234
16. Mihalik S J, Goodpaster B H, Kelley D E, Chace D H, Vockley J, Toledo F G, DeLany J P: Increased levels of plasma acylcarnitines in obesity and type 2 diabetes and identification of a marker of glucolipotoxicity. Obesity 2010; 18:1695-1700
17. Suhre K, Meisinger C, Doring A, Altmaier E, Belcredi P, Gieger C, Chang D, Milburn M V, Gall W E, Weinberger K M, Mewes H W, Hrabe de Angelis M, Wichmann H E, Kronenberg F, Adamski J, Illig T: Metabolic footprint of diabetes: a multiplatform metabolomics study in an epidemiological setting. PloS one 2010; 5:e13953
18. Wang T J, Larson M G, Vasan R S, Cheng S, Rhee E P, McCabe E, Lewis G D, Fox C S, Jacques P F, Fernandez C, O'Donnell C J, Carr S A, Mootha V K, Florez J C, Souza A, Melander O, Clish C B, Gerszten R E: Metabolite profiles and the risk of developing diabetes. Nature medicine 2011; 17:448-453
19. Wang-Sattler R, Yu Z, Herder C, Messias A C, Floegel A, He Y, Heim K, Campillos M, Holzapfel C, Thorand B, Grallert H, Xu T, Bader E, Huth C, Mittelstrass K, Doring A, Meisinger C, Gieger C, Prehn C, Roemisch-Margl W, Carstensen M, Xie L, Yamanaka-Okumura H, Xing G, Ceglarek U, Thiery J, Giani G, Lickert H, Lin X, Li Y, Boeing H, Joost H G, de Angelis M H, Rathmann W, Suhre K, Prokisch H, Peters A, Meitinger T, Roden M, Wichmann H E, Pischon T, Adamski J, Illig T: Novel biomarkers for pre-diabetes identified by metabolomics. Molecular systems biology 2012; 8:615
20. Ha C Y, Kim J Y, Paik J K, Kim O Y, Paik Y H, Lee E J, Lee J H: The association of specific metabolites of lipid metabolism with markers of oxidative stress, inflammation and arterial stiffness in men with newly diagnosed type 2 diabetes. Clinical endocrinology 2012; 76:674-682
21. Floegel A, Stefan N, Yu Z, Muhlenbruch K, Drogan D, Joost H G, Fritsche A, Haring H U, Hrabe de Angelis M, Peters A, Roden M, Prehn C, Wang-Sattler R, Illig T, Schulze M B, Adamski J, Boeing H, Pischon T: Identification of serum metabolites associated with risk of type 2 diabetes using a targeted metabolomic approach. Diabetes 2013; 62:639-648
22. Prentice K J, Luu L, Allister E M, Liu Y, Jun L S, Sloop K W, Hardy A B, Wei L, Jia W, Fantus I G, Sweet D H, Sweeney G, Retnakaran R, Dai F F, Wheeler M B: The furan fatty acid metabolite CMPF is elevated in diabetes and induces beta cell dysfunction. Cell metabolism 2014; 19:653-666
23. Lappas M, Mundra P A, Wong G, Huynh K, Jinks D, Georgiou H M, Permezel M, Meikle P J: The prediction of type 2 diabetes in women with previous gestational diabetes mellitus using lipidomics. Diabetologia 2015; 58:1436-1442
24. Gunderson E P, Matias S L, Hurston S R, Dewey K G, Ferrara A, Quesenberry C P, Jr., Lo J C, Sternfeld B, Selby J V: Study of Women, Infant Feeding, and Type 2 diabetes mellitus after GDM pregnancy (SWIFT), a prospective cohort study: methodology and design. BMC public health 2011; 11:952-963
25. Gunderson E P, Hedderson M M, Chiang V, Crites Y, Walton D, Azevedo R A, Fox G, Elmasian C, Young S, Salvador N, Lum M, Quesenberry C P, Lo J C, Sternfeld B, Ferrara A, Selby J V: Lactation intensity and postpartum maternal glucose tolerance and insulin resistance in women with recent GDM: the SWIFT cohort. Diabetes Care 2012; 35:50-56
26. Gunderson E P K C, Quesenberry C P, Jr., Marcovina S, Walton D, Azevedo R A, Fox G, Elmasian C, Young S, Salvador N, Lum M, Crites Y, Lo J C, Ning X, Dewey K G: Lactation intensity and fasting plasma lipids, lipoproteins, non-esterified free fatty acids, leptin and adiponectin in postpartum women with recent gestational diabetes mellitus: the SWIFT cohort. Metabolism. 2014; 63:941-950
27. Gunderson E P, Hurston S R, Ning X, Lo J C, Crites Y, Walton D, Dewey K G, Azevedo R A, Young S, Fox G, Elmasian C C, Salvador N, Lum M, Sternfeld B, Quesenberry C P, Jr., Study of Women Infant Feeding and Type 2 Diabetes After GDM Pregnancy Investigators. Lactation and Progression to Type 2 Diabetes Mellitus After Gestational Diabetes Mellitus: A Prospective Cohort Study. Ann Intern Med. 2015; 163:889-898
28. Wang T J, Ngo D, Psychogios N, Dejam A, Larson M G, Vasan R S, Ghorbani A, O'Sullivan J, Cheng S, Rhee E P, Sinha S, McCabe E, Fox C S, O'Donnell C J, Ho J E, Florez J C, Magnusson M, Pierce K A, Souza A L, Yu Y, Carter C, Light P E, Melander O, Clish C B, Gerszten R E: 2-Aminoadipic acid is a biomarker for diabetes risk. The Journal of clinical investigation 2013; 123:4309-4317
29. Tan B, Liang Y, Yi L, Li H, Zhou Z, Ji X, Deng J: Identification of free fatty acids profiling of type 2 diabetes mellitus and exploring possible biomarkers by GC-MS coupled with chemometrics. Metabolomics 2010; 6:219-228
30. Kim S, Kim J, Zhang B T: Ensembled support vector machines for human papillomavirus risk type prediction from protein secondary structures. Computers in biology and medicine 2009; 39:187-193
31. Metzger B E, Cho N H, Roston S M, Radvany R: Prepregnancy weight and antepartum insulin secretion predict glucose tolerance five years after gestational diabetes mellitus. Diabetes Care 1993; 16:1598-1605
32. Aroda V R, Christophi C A, Edelstein S L, Zhang P, Herman W H, Barrett-Connor E, Delahanty L M, Montez M G, Ackermann R T, Zhuo X, Knowler W C, Ratner R E, Diabetes Prevention Program Research G: The effect of lifestyle intervention and metformin on preventing or delaying diabetes among women with and without gestational diabetes: the Diabetes Prevention Program outcomes study 10-year follow-up. The Journal of clinical endocrinology and metabolism 2015; 100:1646-1653
33. Floyd J C, Jr., Fajans S S, Conn J W, Knopf R F, Rull J: Stimulation of insulin secretion by amino acids. The Journal of clinical investigation 1966; 45:1487-1502
34. Newgard C B, An J, Bain J R, Muehlbauer M J, Stevens R D, Lien L F, Haqq A M, Shah S H, Arlotto M, Slentz C A, Rochon J, Gallup D, Ilkayeva O, Wenner B R, Yancy W S, Jr., Eisenson H, Musante G, Surwit R S, Millington D S, Butler M D, Svetkey L P: A branched-chain amino acid-related metabolic signature that differentiates obese and lean humans and contributes to insulin resistance. Cell metabolism 2009; 9:311-326
35. Menni C, Fauman E, Erte I, Perry J R, Kastenmuller G, Shin S Y, Petersen A K, Hyde C, Psatha M, Ward K J, Yuan W, Milburn M, Palmer C N, Frayling T M, Trimmer J, Bell J T, Gieger C, Mohney R P, Brosnan M J, Suhre K, Soranzo N, Spector T D: Biomarkers for type 2 diabetes and impaired fasting glucose using a nontargeted metabolomics approach. Diabetes 2013; 62:4270-4276
36. Samuel V T: Fructose induced lipogenesis: from sugar to fat to insulin resistance. Trends in endocrinology and metabolism: TEM 2011; 22:60-65
37. Maedler K, Spinas G A, Dyntar D, Moritz W, Kaiser N, Donath M Y: Distinct effects of saturated and monounsaturated fatty acids on beta-cell turnover and function. Diabetes 2001; 50:69-76
38. Peter Slotte J: Molecular properties of various structurally defined sphingomyelins—correlation of structure with function. Progress in lipid research 2013; 52:206-219
39. Anderson S G, Dunn W B, Banerjee M, Brown M, Broadhurst D I, Goodacre R, Cooper G J, Kell D B, Cruickshank J K: Evidence that multiple defects in lipid regulation occur before hyperglycemia during the prodrome of type-2 diabetes. PloS one 2014; 9:e103217
40. Wang Q, Imamura F, Ma W, Wang M, Lemaitre R N, King I B, Song X, Biggs M L, Delaney J A, Mukamal K J, Djousse L, Siscovick D S, Mozaffarian D: Circulating and dietary trans Fatty acids and incident type 2 diabetes in older adults: the cardiovascular health study. Diabetes Care 2015; 38:1099-1107
41. Mai M, Tonjes A, Kovacs P, Stumvoll M, Fiedler G M, Leichtle A B: Serum levels of acylcarnitines are altered in prediabetic conditions. PloS one 2013; 8:e82459
42. Waljee A K, Higgins P D, Singal A G: A primer on predictive models. Clinical and translational gastroenterology 2014; 5:e44

We claim:

1. A method of predicting progression of gestational diabetes (GDM) to Type 2 diabetes (T2D) in a subject, the method comprising:
analyzing a biological sample of a subject to determine levels of a plurality of metabolites in the sample, wherein the plurality of metabolites comprises one or more of PCaeC40:5 and SM(OH)C14:1 and at least two metabolites set forth in Table 3, 4 and/or 6; and
comparing the determined levels of the plurality of metabolites in the sample to a corresponding plurality of reference levels in order to predict progression of GDM to T2D in the subject.

2. The method of claim 1, wherein the plurality of metabolites comprises at least one amino selected from: 2-Aminoadipic acid, Gly, Arg, Gln, His, Ile, Leu, Met, Orn, Phe, PAG, Pro, Ser, Thr, Trp, Tyr, Val, and xLeu.

3. The method of claim 2, wherein the amino acid is one or more branched chain amino acid selected from: 2-Aminoadipic acid, Gly, Ile, Leu, Thr, Trp, Tyr, Val, xLeu, preferably xLeu and Val.

4. The method of any one of claims 1-3, wherein the plurality of metabolites comprises at least one sphingomyelin (SM) species selected from: SM(OH)C14:1, SM (OH) C16:1, SM (OH) C22:1, SM (OH) C22:2, SM (OH) C24:1, SM C16:0, SM C16:1, SM C18:0, SM C18:1, SM C20:2, SM C24:0, SM C24:1, preferably SM(OH)C14:1.

5. The method of any one of claims 1-4, wherein the plurality of metabolites comprises at least one lipid/fatty acid selected from: Myristic acid (C14:0), Palmitic acid (C16:0), Hexadecenoic acid (C16:1 n-7), Palmitoleic acid (C16:1 n-9), Stearic acid (C18:0), Oleic Acid & Vaccenic Acid (C18:1 n-9, n-7), Linoleic acid (C18:2), Alpha-linolenic acid (C18:3), Eicosenoic acid (C20:1), Arachidonic acid (C20:4), Eicosapentaenoic acid (C20:5), Docosapentaenoic acid (C22:5), Docosahexaenoic acid (C22:6), preferably, Palmitoleic acid (C16:1 n9).

6. The method of any one of claims 1-5, wherein the plurality of metabolites comprises at least one ketone, preferably beta-hydroxybutyrate.

7. The method of claim 1, wherein the plurality of metabolites comprises one or more of PCaeC40:5 and SM(OH)C14:1, and two or more of 2-aminoadipic acid, Ile, Leu, Thr, Trp, Tyr, Val, xLeu, Hexose, AC3, Gly, SM (OH) C16:1, SM (OH) C22:2, SM C18:0, SM C18:1, SM C20:2, SM C24:1, PC ae C42:5, PC ae C44:5, AC10, and palmitoleic acid (C16:1 n9).

8. The method of any one of claims 1 to 7, wherein the plurality of reference levels are indicative of levels of the plurality of metabolites in subjects whose GDM did not progress to T2D.

9. The method of claim 8, wherein a determined increase of one or more of 2-aminoadipic acid, Ile, Leu, Thr, Trp, Tyr, Val, xLeu, Hexose and AC3 relative to the respective plurality of reference levels is indicative of progression of GDM to T2D.

10. The method of claim 8 or 9, wherein a determined decrease of one or more of Gly, SM(OH)C14:1, SM (OH) C16:1, SM (OH) C22:2, SM C18:0, SM C18:1, SM C20:2, SM C24:1, PC ae C40:5, PC ae C42:5, PC ae C44:5, AC10 and palmitoleic acid (C16:1 n9) relative to the respective plurality of reference levels is indicative of progression of GDM to T2D.

11. The method of any one of claims 7 to 10, wherein the plurality of metabolites comprises PC ae C40:5, SM (OH) C14:1, hexoses, Val, Leu, and Ile.

12. The method of any one of claims 1 to 11, wherein the progression of GDM to T2D is within 0-5 years of delivery, preferably within 0-2 years of delivery.

13. The method of any one of claims 1 to 12, wherein the biological sample comprises a plasma sample, preferably a fasting plasma sample.

14. The method of claim 13, wherein the plasma sample is obtained from the subject at 6-9 weeks post-partum.

15. The method of any one of claims 1 to 14, wherein the determining is by one or more of LC-MS/MS, GC-MS, ELISA while Fasting (FPG) and antibody detection.

16. The method of any one of claims 1 to 15, further comprising:
treating the subject based on a result of the comparison, wherein the treatment comprises one or more of: diet regimen, exercise regimen, blood sugar monitoring, insulin therapy, and medication.

17. The method of any one of claims 1 to 16, wherein the determination of the levels of the plurality of metabolites in the sample comprises detecting a derivative of one or more of the plurality of metabolites.

18. A computer-implemented method of predicting progression of gestational diabetes (GDM) to Type 2 diabetes (T2D) in a subject comprising:
measuring in a biological sample from a post-partum subject an incident type 2 diabetes (T2D) biomarker panel, wherein the incident T2D biomarker panel comprises one or more of PCaeC40:5 and SM(OH)C14:1 and at least two biomarkers set forth in Table 3, 4 and/or 6;
applying the measured incident T2D biomarker panel from the subject against a database of measured T2D biomarker panels from control subjects, wherein the database is stored on a computer system; and
determining that the subject has an increased risk of progression to T2D by measuring difference in the incident T2D biomarker panel relative to measured incident T2D biomarker panels from control subjects.

19. A non-transitory computer readable storage medium with an executable program stored thereon, wherein the program comprises instructions for evaluating a subject's risk for progressing from GDM to T2D, and wherein the program instructs a microprocessor to perform one or more of the steps of any one of the methods of claims 1-18.

20. A computer system comprising:
a database including records comprising reference metabolite profiles associated with clinical outcomes, each reference profile comprising the levels of a set of metabolites listed in Table 3, 4, and/or 6;
a user interface capable of receiving and/or inputting a selection of metabolite levels of a set of metabolites, the set of metabolites listed in Table 3, 4, and/or 6 for use in comparing to the metabolite reference profiles in the database; and
an output that displays a prediction of clinical prognosis according to the levels of the set of metabolites.

21. The computer system of claim 20 for performing a method of any one of claims 1 to 18.
